SEQUENCE LISTING (6 sequences)

SEQ ID NO: 1 - DNA, 21 bp, Homo sapiens
aagagctgcaaggccttgccc

SEQ ID NO: 2 - DNA, 21 bp, Homo sapiens
aacctcatccagacagaacga

SEQ ID NO: 3 - DNA, 20 bp, Artificial Sequence
  Description of Artificial Sequence Synthetic oligonucleotide
aatctccgaacgtgtcacgt

SEQ ID NO: 4 - protein, 6 aa, Artificial Sequence
  Description of Artificial Sequence Synthetic peptide
TFLLRN

SEQ ID NO: 5 - protein, 6 aa, Artificial Sequence
  Description of Artificial Sequence Synthetic peptide
SLIGRL

SEQ ID NO: 6 - protein, 6 aa, Artificial Sequence
  Description of Artificial Sequence Synthetic peptide
AYPGKF

(19) United States
(12) Patent Application Publication
(10) Pub. No.: US 2006/0116343 A1
(43) Pub. Date: Jun. 1, 2006

(54) Sphingosine kinase-1 mediates gene expression regulation of a monocyte chemoattractant protein-1 gene

(76) Inventors: Alejandro Bernal, North Wales, PA (US); Claudia K. Derian, Hatboro, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/274,971

(22) Filed: Nov. 16, 2005

Related U.S. Application Data
(60) Provisional application No. 60/628,390, filed on Nov. 16, 2004.

(51) Int. Cl. A61K 48/00; C12Q 1/68; C12Q 1/48

(52) U.S. Cl. 514/44; 435/6; 435/15

(57) ABSTRACT

The present invention relates to methods of identifying, monitoring, and using compounds that regulate the biological activity of Sphingosine kinase-1.

### CROSS REFERENCE TO RELATED APPLICATIONS

This application claims priority to Application No. 60/628,390 filed on Nov. 16, 2004.

### FIELD OF THE INVENTION

The present invention relates to methods of identifying, monitoring, and using compounds that regulate the biological activity of Sphingosine kinase-1. In particular, methods of the invention relates to signal transductions involving Sphingosine kinase-1, thrombin, or monocyte chemoattractant protein-1.

### BACKGROUND OF THE INVENTION

The vascular endothelium, once thought to provide a passive barrier function between the blood-tissue compartments, is now well recognized to serve a prominent role in maintaining normal hemostasis and coordinating the tissue response to injury. Increased exposure of the endothelium to a wide spectrum of stimuli occurs during periods of acute injury as in mild inflammatory or thrombotic episodes when the vasculature comes in contact with cellular elements of the blood as well as proteins released as a consequence of degranulation. Under conditions of more sustained activation of the vasculature as is considered to occur in diabetes or atherosclerosis, exposure to elevated lipids, hyperglycemia and changes in shear stress present yet another level of complexity in the interaction of stimuli regulating endothelium pathophysiology. The stimulation of endothelium results in release of numerous bioactive mediators as well as changes in endothelial surface molecules which promote platelet and leukocyte adherence and emigration, changes in vascular integrity and vascular reactivity.

Sphingosine kinases (SKs) are a recently discovered family of lipid kinases evolutionarily conserved in humans, mice, yeast, and plants (Nava et al., FEBS Lett. 2000, 473:81-84). Two SK isotypes, SK1 and SK2, have recently been cloned from human and mouse (Liu et al., J Biol. Chem. 2000, 275(26):19513-20). SK1 and SK2 differ with respect to kinetic properties, tissue distribution and developmental expression patterns, suggesting potentially distinct regulatory and functional roles (Fukuda et al., Biochem Biophys Res Commun., 2003, 309:155-160). SKs are expressed in many cell types including human vascular endothelial cells and vascular smooth muscle cells (VSMCs) (Xu et al., Atherosclerosis 2002, 164:237-43; Ren et al., World J. Gastroenterol. 2002, 8:602-7). SK catalyzes the formation of sphingosine-1-phosphate (S1P) from sphingosine.

S1P is a bioactive lipid that regulates, both extracellularly and intracellularly, diverse biological processes. For example, S1P alleviates the generation of cytotoxic ceramide, known to be a potent inducer of programmed cell death, or apoptosis (Maceyka et al., Biochimica et Biophysica Acta (BBA)—Molecular and Cell Biology of Lipids, 2002, 1585:193-201). Moreover, SIP has been shown to directly or indirectly play a role in the release of intracellular calcium stores in an inositol-triphosphate (IP3)-independent manner (Mattie et al., J. Biol. Chem. 1994, 269:3181-3188), activation of transcription factors such as CREB (Coussin et al., Biochem Pharmacol, 2003, 66:1861-1870) and AP-1 (Su et al., J Biol Chem 1994, 269:16512-16517), increase of the expression of inflammation-related proteins (Xia et al., Proc Natl Acad Sci USA, 1998, 95:14196-14201), and stimulation of mitogenesis (Auge et al., J Biol Chem. 1999, 274:21533-21538). Most recently, S1P signaling has been suggested to play an important role in the pathogenesis of atherosclerotic lesions, although it is yet to be investigated whether S1P is atherogenic or anti-atherogenic (Xu et al., Acta Pharmacol Sin 2004, 25:849-954).

The activation of SK has been described downstream of several diverse receptor families in numerous cell types. For example, SK is involved in the signaling pathway mediated by the proinflammatory cytokine receptors such as TNF-α receptor (Xia et al., supra). SK is also involved in the signaling pathways of other receptor families, such as receptor tyrosine kinases (i.e. VEGF or PDGF receptors) (Wu et al., Oncogene 2003, 22:3361-3370; Meyer zu Heringdorf et al., FEBS Letters 1999, 461:217-222; and Hobson et al., Science 2001, 291:1800-1803), high affinity FcεRI receptors (Choi et al., Nature 1996, 380:634-636), and GPCRs (i.e. muscarinic (Meyer zu Heringdorf, et al., supra), and S1P-receptors).

Thrombin is a trypsin like serine protease fulfilling a central role in both haemostasis and thrombosis (see review Srivastava et al., Med Res Rev. 2005 January; 25(1):66-92). Thrombosis is the most common singular cause of death in the developed countries, which is typified by abnormal coagulation and platelet aggregation. Some debilitating indications manifest themselves in the form of myocardial infarction, stroke, deep vein thrombosis, and pulmonary embolism. In fact, the American Heart Association estimated that 54% of all deaths in the US can be attributed to cardiovascular diseases. Thrombosis related complications account for approximately 2 million deaths alone in the US every year. Many if not most, episodes of thrombosis can be prevented by use of an appropriate primary antithrombotic therapy and almost all instances of recurrence can be prevented by use of an appropriate secondary therapy. Haemostasis, which is a complex process that defends against uncontrolled hemorrhage in the event of damage to blood vessels, can be activated either by vessel injury, tissue injury, or the presence of foreign bodies in the blood stream. The haemostatic mechanism of action involves: (a) Vasospasm of injured vessel; (b) Formation of a short term platelet plug; (c) Formation of a strong fibrin clot (thrombus); and (d) Dissolution of the clot (fibrinolysis).

Thrombin is known for its primary function in the maintenance of hemostasis through its well-characterized role in the coagulation pathway. Thrombin proteolytically cleaves fibrinogen to fibrinopeptides A and B and generates fibrin. Fibrin forms the fibrin clot (thrombus) that prevents blood loss after vascular injury. The clot is subsequently removed by the fibrinolytic system (fibrinolysis) upon wound healing. Apart from its key role at the final step of the coagulation process, i.e., the process of forming clot, thrombin also plays a key role in the initiation of the inhibitory pathways to down-regulate the coagulation process and activate the fibrinolytic system. An increased activation of coagulation can result in severe thromboembolic disorders, such as thrombosis.

Thrombin is also implicated as a key mediator in the cellular response to tissue injury through activation of platelets, vascular cells, fibroblasts and immune cells through a novel family of seven transmembrane G-protein-coupled receptors known as protease-activated receptors (PAR). (Triplett, Clin Chem. 2000, 46:1260-1269). Thrombin induces the expression of adhesion molecules, increases vascular permeability, angiogenesis and the release of cytokines and growth factors, and promotes the release of vasoregulators such as nitric oxide and prostaglandins. Most of the cellular responses of thrombin have been attributed to PAR-1 (Derian et al., EXPERT OPINION ON INVESTIGATIONAL DRUGS, 2003, 12(2): 209-21). However, two additional thrombin responsive receptors have been identified, PAR-3 (Ishihara et al., Nature, 1997, 386:502-506) and PAR-4 (Xu et al., PNAS 1998, 95: 6642-6646). A thrombin insensitive receptor within this GPCR family, PAR-2, has also been identified, which is expressed on vascular endothelium (Bohm et al., Biochem J 1996, 314 (Pt 3:1009-1016) and upon activation mediates many parallel biological processes as PAR-1. Inhibitors for the thrombin signaling pathway have become a major focus for the current investigation of therapeutics for thrombosis and other diseases.

Various reports suggested that SK is either involved or not involved in thrombin signaling pathway in various cell types. For example, it was reported that activation of SK is involved in thrombin induced IL-6 secretion by mouse mast cell (Gordon et al., Cell Immunol. 2000 Nov. 1; 205(2):128-35). However, in intact human platelets labeled with [³H]-sphingosine, stimulation with thrombin did not affect [³H]-S1P formation (Yang et al., J Biochem (Tokyo). 1999 July; 126(1):84-9). Most recently, it was reported that in myenteric glia of the guinea pig, activation of PAR-2, a GPCR that is activated by trypsin and mast cell tryptase, but not thrombin, leads to increases in intracellular calcium via a signal transduction mechanism that involves activation of sphingosine kinase (Garrido et al., J. Neurochem. 2002 November; 83(3):556-64). Interestingly, the same report showed that in myenteric glia of the guinea pig, activation of PAR-1, a GPCR that is activated by thrombin, leads to increases in intracellular calcium via a signal transduction mechanism that does not involve activation of sphingosine kinase, but activation of phospholipase C instead.

Monocyte chemoattractant protein-1 (MCP-1) is a C—C chemokine that plays a critical role in the recruitment of monocytes, macrophages, and T lymphocytes under both physiological and pathophysiological conditions. The chemotactic effects of MCP-1 are mediated by the chemokine receptor CCR2 (Boring et al., J. Clin. Invest. 1997, 100: 2552-2561). MCP-1 is expressed in various tissues such as endothelial, bronchial, epithelial, smooth muscle cells, macrophages, etc. (Antoniades et al, Proc Natl Acad Sci USA, 1992, 89:5371-5375; Lyonaga et al., Hum Pathol, 1994, 25:455-463; Car et al, Am J Respir Crit Care Med, 1994, 149:655-659).

MCP-1 is implicated in the pathogenesis of various inflammatory diseases involving monocyte/macrophage infiltration of the affected tissue. Levels of MCP-1 are elevated in synovial fluid and serum of patients with rheumatoid arthritis and in synovial fluid of rats with collagen-induced arthritis (CIA) (Koch et al., J. Clin. Invest. 90, 1992: 772-779; Ogata et al., J. Pathol. 1997, 182: 106-114). Data from animal models support an essential role of MCP-1 in the development of rheumatoid arthritis. For example, in rats with CIA, administration of a neutralizing monoclonal antibody against MCP-1 reduces joint swelling and macrophage invasion; in the mouse MRL-lpr model of arthritis, injection of a dominant-negative form of MCP-1 prevents the onset of joint inflammation and bone destruction (Ogata et al., supra; and Gong et al., J. Exp. Med. 1997, 186:131-137). MCP-1 is a potential therapeutic target for the treatment of several inflammatory conditions, including rheumatoid arthritis and chronic obstructive pulmonary disease.

Recent studies have revealed that increased expression of MCP-1 plays a central role in the pathogenesis of vascular diseases (Charo et al., Circulation Research. 2004; 95: 858). MCP-1 has been implicated as a key player in the recruitment of monocytes from the blood into early atherosclerotic lesions, the development of intimal hyperplasia after angioplasty, as well as in vasculogenesis and in aspects of thrombosis. Transgenic mice lacking either MCP-1 or CCR2 show a partial resistance to experimentally induced atherosclerosis (see for example, Yla-Herttuala et al., Proc. Natl. Acad. Sci. USA. 1991, 88: 5252-5256; Boring et al., Nature, 1998, 394: 894-897).

In addition, studies have associated MCP-1 with chronic obstructive pulmonary disease (de Boer et al., Pathol. 2000, 190: 619-626; Traves et al., 2002, Thorax 57: 590-595) and ischemic tissue damage following stroke (Hughes et al., J. Cereb. Blood Flow Metab. 2002, 22: 308-317).

Compounds designed to inhibit the biological activity of MCP-1 may offer therapeutic benefit in a number of disease areas. Anti-MCP-1 gene therapy has been suggested as a useful and feasible strategy against MCP-1 related cardiovascular diseases (Kitamoto et al., Expert Rev Cardiovasc Ther. 2003 September; 1(3): 393-400). For example, transfecting skeleton muscles with a dominant negative inhibitor of MCP-1, suppressed arteriosclerotic changes induced by chronic inhibition of nitric oxide synthesis in rats. Such a gene therapy inhibited the development, progression and destabilization of atherosclerosis in apolipoprotein E knockout mice. This strategy also reduced restenosis after balloon injury in rats, rabbits and monkeys, and reduced neointimal formation after stent implantation in rabbits and monkeys (Kitamoto, supra).

Other biological agents, including antibodies and inhibitory peptides, have also been developed for treating diseases related to MCP-1. For example, a monoclonal antibody that blocks the binding of MCP-1 to CCR2 is being used in phase II trials for rheumatoid arthritis (Charo et al. supra). However, despite intensive screening, there still lacks small-molecule antagonists of the receptor of CCR2 that can be used clinically (Daly et al., Microcirculation. 2003 June; 10(3-4): 247-57). Therefore, there is a need for new strategies to develop new therapeutic agents for the treatment of MCP-1 related diseases.

### SUMMARY OF THE INVENTION

It is now discovered that sphingosine kinase-1 (SK1) is activated by thrombin via Par-1 and by tumor necrosis factor alpha. Activation of SK1 by either thrombotic or inflammatory stimuli induces expression of a monocyte chemoattractant protein-1 (MCP-1) gene. Reducing SK1 activity, either by an inhibitor of the SK1 activity or by a siRNA that specifically decreases the expression of the SK1 gene, inhibits the SK1 induced expression of the MCP-1 gene.

In one general aspect, the present invention provides a method of determining a biological activity of a sphingosine kinase-1 in a cell, comprising the step of determining the expression level of a monocyte chemoattractant protein-1 gene from the cell.

In another general aspect, the present invention provides a method of monitoring the effectiveness of a compound administered to a subject, wherein said compound is expected to increase or decrease the biological activity of a sphingosine kinase-1 in a cell of said subject, comprising the steps of: a) measuring the expression level of a monocyte chemoattractant protein-1 gene from said subject; and b) comparing the expression level determined in step a) with the expression level of a monocyte chemoattractant protein-1 gene in the subject prior to the administration of said compound. In a particular embodiment, step (a) of the method comprises the step of measuring the amount of a monocyte chemoattractant protein-1 protein in a biological sample of the subject.

Another general aspect of the invention is a method of identifying a compound that increases or decreases the biological activity of a sphingosine kinase-1, comprising the steps of: a) contacting a sphingosine kinase-1-responsive system with a solution comprising a buffer and a test compound, wherein the sphingosine kinase-1-responsive system comprises a sphingosine kinase-1 or a functional derivative thereof, and a gene whose expression is controlled by a regulatory sequence of a monocyte chemoattractant protein-1 gene; b) measuring from the sphingosine kinase-1-responsive system the expression level of the gene whose expression is controlled by a regulatory sequence of a monocyte chemoattractant protein-1 gene; and c) identifying the compound by its ability to increase or decrease said expression level as compared to a control wherein the sphingosine kinase-1-responsive system is contacted with only the buffer.

In a particular embodiment to this aspect, the method further comprises the steps of: d) contacting a sphingosine kinase-1 with a solution comprising the compound identified from step c) above and a buffer comprising sphingosine and adenosine triphosphate; e) measuring the amount of sphingosine-1-phosphate produced from the sphingosine; and f) confirming the compound by its ability to increase or decrease the production of sphingosine-1-phosphate from the sphingosine as compared to a control wherein the sphingosine kinase-1 is contacted with only the buffer.

Another general aspect of the invention is a method of increasing or decreasing expression of a monocyte chemoattractant protein-1 gene in a cell, comprising the step of increasing or decreasing the biological activity of a sphingosine kinase-1 in the cell such that expression of said monocyte chemoattractant protein-1 gene is increased or decreased, respectively.

Another general aspect of the invention is a method of inhibiting thrombin signal transduction in a cell, comprising the step of decreasing the biological activity of a sphingosine kinase-1 in the cell such that said thrombin signal transduction is inhibited.

The present invention further provides methods of treating or preventing a disease related to the thrombin signal transduction pathway or a disease related to increased MCP-1 biological activity or gene expression in a subject. Such methods comprise the step of decreasing the biological activity of a sphingosine-1-phosphate in the subject such that the disease is treated or prevented. In particular embodiments, such a disease is thrombosis or atherosclerosis.

### BRIEF DESCRIPTION OF THE DRAWINGS

FIG. 1 illustrates the results from the Microarray analyses. The analyses uncovered linkage of thrombin signal transduction and sphingosine kinase, that MCP-1 gene expression was induced in response to both stimuli, thrombin and TNF-α, and that the induction was inhibited in conditions where DMS was added prior to stimulation.

FIG. 2 illustrates that the designed siRNAs showed specificity for their respective human SK isoforms as detected by Taqman quantitative RT-PCR.

FIG. 3 illustrates that specific inhibition of SK-1 abrogates induction of MCP-1 by TNFα or thrombin: the induction of MCP-1 was inhibited in the presence of hSK1-specific siRNAs, but not hSK2-specific or control non-silencing siRNAs.

FIG. 4 illustrates that secretion of MCP-1 is a PAR-1 dependent mechanism and requires sphingosine kinase activity.

FIG. 5 illustrates that PAR-1 mediated induction of MCP-1 by thrombin requires hSK1, but not hSK2: the induction of MCP-1 mediated by thrombin/PAR-1 was inhibited in the presence of hSK1-specific siRNAs, but not hSK2-specific or control non-silencing siRNAs.

FIG. 6 illustrates that induced MCP-1 secretion from HMVECs requires NF-κB activity.

FIG. 7 illustrates that induction of MCP-1 is not mediated by activation of S1P receptors.

### DETAILED DESCRIPTION OF THE INVENTION

All publications cited hereinafter are hereby incorporated by reference. Unless defined otherwise, all technical and scientific terms used herein have the same meaning as commonly understood to one of ordinary skill in the art to which this invention pertains.

As used herein, the terms “comprising”, “containing”, “having” and “including” are used in their open, non-limiting sense.

The following are some abbreviations that are at times used in this specification:

ATP=adenosine triphosphate;

bp=base pair;

cDNA=complementary DNA;

DMS=dimethylsphingosine;

ELISA=enzyme-linked immunoabsorbent assay;

FLIPR=fluorescence imaging plate reader;

GPCR=G protein coupled receptor;

hSK=human sphingosine kinase

kb=kilobase; 1000 base pairs;

MCP-1=Monocyte chemoattractant protein-1;

NF-kB=Nuclear factor kappa B;

nt=nucleotide;

PAGE=polyacrylamide gel electrophoresis;

PAR=protease-activated receptors;

PCR=polymerase chain reaction;

RT-PCR=Reverse transcription polymerase chain reaction;

S1P=Sphingosine-1-phosphate;

SDS=sodium dodecyl sulfate;

siRNA=Small interference RNA;

SSC=sodium chloride/sodium citrate;

SK1=sphingosine kinase-1;

TNFα=Tumor necrosis factor alpha; and

UTR=untranslated region.

“An activity”, “a biological activity”, or “a functional activity” of a polypeptide or nucleic acid refers to an activity exerted by a polypeptide or nucleic acid molecule as determined in vivo, or in vitro, according to standard techniques. Such an activity can be a direct activity such as an ion channel activity. It can also be an association with or an enzymatic activity on a second protein or substrate, for example, the serine protease activity of a thrombin or the lipid kinase activity of a SK. A biological activity of protein can also be an indirect activity, such as a cellular signaling activity mediated by interaction of the protein with one or more than one additional protein or other molecule(s), including but not limited to, interactions that occur in a multi-step, serial fashion.

A “biological sample” as used herein refers to a sample containing or consisting of cell or tissue matter, such as cells or biological fluids isolated from a subject. The “subject” can be a mammal, such as a rat, a mouse, a monkey, or a human, that has been the object of treatment, observation or experiment. Examples of biological samples include, for example, sputum, blood, blood cells (e.g., white blood cells), amniotic fluid, plasma, semen, bone marrow, tissue or fine-needle biopsy samples, urine, peritoneal fluid, pleural fluid, and cell cultures. Biological samples may also include sections of tissues such as frozen sections taken for histological purposes. A test biological sample is the biological sample that has been the object of analysis, monitoring, or observation. A control biological sample can be either a positive or a negative control for the test biological sample. Often, the control biological sample contains the same type of tissues, cells and/or biological fluids of interest as that of the test biological sample.

In particular embodiments, the biological sample is a “clinical sample,” which is a sample derived from a human patient. A biological sample may also be referred to as a “patient sample.” A test biological sample is the biological sample that has been the object of analysis, monitoring, or observation. A control biological sample can be either a positive or a negative control for the test biological sample. Often, the control biological sample contains the same type of tissues, cells and biological fluids of interest as that of the test biological sample.

A “cell” refers to at least one cell or a plurality of cells appropriate for the sensitivity of the detection method. Cells suitable for the present invention may be bacterial, but are preferably eukaryotic, and are most preferably mammalian. An “endothelial cell” is a thin, flattened cell that can be found as a layer inside surfaces of body cavities, blood vessels, and lymph vessels making up the endothelium. Cell lines of “endothelial cell” have been established that can be maintained in culture media in vitro. Examples of endothelial cell lines, include, but are not limited to, adult human microvascular endothelial cells (HMVECs), HUV-EC-C, human aortic endothelial cells (HAEC).

A “clone” is a population of cells derived from a single cell or common ancestor by mitosis. A “cell line” is a primary cell that derives clonal expansion of cells and is capable of stable growth in vitro for many generations.

A “gene” is a segment of DNA involved in producing a peptide, polypeptide, or protein, and the mRNA encoding such protein species, including the coding region, non-coding regions preceding (“5′UTR”) and following (“3′UTR”) the coding region. A “gene” may also include intervening non-coding sequences (“introns”) between individual coding segments (“exons”). “Promoter” means a regulatory sequence of DNA that is involved in the binding of RNA polymerase to initiate transcription of a gene. Promoters are often upstream (“5′ to”) the transcription initiation site of the gene. A “regulatory sequence” refers to the portion of a gene that can control the expression of the gene. A “regulatory sequence” can include promoters, enhancers and other expression control elements such as polyadenylation signals, ribosome binding site (for bacterial expression), and/or, an operator. An “enhancer” means a regulatory sequence of DNA that can regulate the expression of a gene in a distance- and orientation-dependent fashion. A “coding region” refers to the portion of a gene that encodes amino acids and the start and stop signals for the translation of the corresponding polypeptide via triplet-base codons.

“Gene expression microarray analysis” refers to an assay wherein a “microarray” of probe oligonucleotides is contacted with a nucleic acid sample of interest, e.g., a target sample, such as poly A mRNA from a particular tissue type, or a reverse transcript thereof. See, e.g., Nees et al: (2001), Curr Cancer Drug Targets, 1(2):155-75. Contact is carried out under hybridization conditions and unbound nucleic acid is removed. The resultant pattern of hybridized nucleic acid provides information regarding the genetic profile of the sample tested. Gene expression analysis can measure expression of thousands of genes simultaneously, providing extensive information on gene interaction and function. Gene expression analysis may find use in various applications, e.g., identifying expression of genes, correlating gene expression to a particular phenotype, screening for disease predisposition, and identifying the effect of a particular agent on cellular gene expression, such as in toxicity testing. “Microarray” as used herein refers to a substrate, e.g., a substantially planar substrate such as a biochip or gene chip, having a plurality of polymeric molecules spatially distributed over, and stably associated with or immobilized on, the surface of the substrate. Exemplary microarray formats include oligonucleotide arrays, and spotted arrays. Methods on gene expression microarray analysis are known to those skilled in the art. See, e.g., review by Yang et al. (2002), Nat Rev Genet 3(8): 579-88), or U.S. Pat. No. 6,004,755, which discloses methods on quantitative gene expression analysis using a DNA microarray.

A “Monocyte chemoattractant protein-1 gene”, “MCP1”, or “MCP-1 gene” each refers to a gene that encodes a monocyte chemoattractant protein-1, and the MCP-1 gene, (1) specifically hybridizes under stringent hybridization conditions to a nucleic acid molecule having greater than about 60% nucleotide sequence identity to the coding region of a human MCP-1 cDNA (NCBI nucleotide accession number: NM_{—}002982); (2) encodes a protein having greater than about 60% amino acid sequence identity to a human MCP-1 protein (NCBI protein accession number: NP_{—}002973); or (3) encodes a protein capable of binding to antibodies, e.g., polyclonal or monoclonal antibodies, raised against the human MCP-1 protein described herein.

The “MCP-1 gene” can specifically hybridize under stringent hybridization conditions to a nucleic acid molecule having greater than about 65, 70, 75, 80, 85, 90, or 95 percent nucleotide sequence identity to the coding region of a human MCP-1 cDNA (NCBI nucleotide accession number: NM_{—}002982). In other embodiments, the MCP-1 gene encodes a protein having greater than about 65, 70, 75, 80, 85, 90, or 95 percent amino acid sequence identity to a human MCP-1 (NCBI protein accession number: NP_{—}002973). Exemplary “MCP-1 gene” includes genes for structural and functional polymorphisms of human MCP-1, and its orthologs in other animals such as rat (i.e., NCBI nucleotide accession NO: NM_{—}031530), mouse (i.e., NCBI nucleotide accession NO: NM_{—}011333), pig, dog and monkey. “Polymorphism” refers to a set of genetic variants at a particular genetic locus among individuals in a population.

A “monocyte chemoattractant protein-1”, “MCP1”, “MCP-1” or “MCP-1 protein” each refers to a protein that is a C—C chemokine, which recruits monocytes, macrophages, or T lymphocytes under both physiological and pathophysiological conditions activation. A MCP-1, (1) has greater than about 60% amino acid sequence identity to a human MCP-1 protein (NCBI protein accession number: NP_{—}002973); (2) binds to antibodies, e.g., polyclonal or monoclonal antibodies, raised against a human MCP-1 (NCBI protein accession number: NP_{—}002973); or (3) is encoded by a polynucleotide that specifically hybridizes under stringent hybridization conditions to a nucleic acid molecule having a sequence that has greater than about 60% nucleotide sequence identity to the coding region of a human MCP-1 cDNA (NCBI nucleotide accession number: NM_{—}002982).

In some embodiments, the MCP-1 has greater than about 65, 70, 75, 80, 85, 90, or 95 percent amino acid sequence identity to a human MCP-1 (NCBI protein accession number: NP_{—}002973). Exemplary MCP-1 includes human MCP-1, which includes structural and functional polymorphisms of the human MCP-1 protein depicted in NCBI protein accession number: NP_{—}002973. MCP-1 also includes orthologs of the human MCP-1 in other animals such as rat (i.e., NCBI nucleotide accession NO: NP_{—}113718), mouse (i.e., NCBI protein accession NO: NP_{—}035463), pig, dog and monkey.

A “sphingosine kinase-1 gene”, “SK1 gene”, or “SphK1 gene”, each refers to a gene that encodes a sphingosine kinase-1, and the SK1 gene, (1) specifically hybridizes under stringent hybridization conditions to a nucleic acid molecule having greater than about 60% nucleotide sequence identity to the coding region of a human SK1 cDNA (NCBI nucleotide accession number: NM_{—}021972); (2) encodes a protein having greater than about 60% amino acid sequence identity to a human SK1 protein (NCBI protein accession number: NP_{—}068807); or (3) encodes a protein capable of binding to antibodies, e.g., polyclonal or monoclonal antibodies, raised against the human SK1 protein described herein.

The “SK1 gene” can specifically hybridize under stringent hybridization conditions to a nucleic acid molecule having greater than about 65, 70, 75, 80, 85, 90, or 95 percent nucleotide sequence identity to the coding region of a human SK1 cDNA (NCBI nucleotide accession number: NM_{—}021972). In other embodiments, the SK1 gene encodes a protein having greater than about 65, 70, 75, 80, 85, 90, or 95 percent amino acid sequence identity to a human SK1 (NCBI protein accession number: NP_{—}068807). Exemplary “SK1 gene” includes genes for structural and functional polymorphisms of human SK1, and its orthologs in other animals including rat (i.e., NCBI nucleotide accession NO: NM_{—}133386), mouse (i.e., NCBI nucleotide accession NO: NM_{—}011451), pig, dog and monkey.

A “sphingosine kinase-1”, “SK1”, “SphK1”, or “SK1 protein” each refers to a protein that upon activation is capable of catalyzing the formation of sphingosine-1-phosphate (S1P) from the lipid sphingosine.

A “SK1”, (1) has greater than about 60% amino acid sequence identity to a human SK1 protein (NCBI protein accession number: NP_{—}068807); (2) binds to antibodies, e.g., polyclonal or monoclonal antibodies, raised against a human SK1 (NCBI protein accession number: NP_{—}068807); or (3) is encoded by a polynucleotide that specifically hybridizes under stringent hybridization conditions to a nucleic acid molecule having a sequence that has greater than about 60% nucleotide sequence identity to the coding region of a human SK1 cDNA (NCBI nucleotide accession number: NM_{—}021972).

In some embodiments, the “SK1” has greater than about 65, 70, 75, 80, 85, 90, or 95 percent amino acid sequence identity to a human SK1 (NCBI protein accession number: NP_{—}068807). Exemplary SK1 includes human SK1, which includes structural and functional polymorphisms of the human SK1 protein depicted in NCBI protein accession number: NP_{—}068807. SK1 also includes orthologs of the human SK1 in other animals such as rat (i.e., NCBI nucleotide accession NO: NP_{—}596877), mouse (i.e., NCBI protein accession NO: NP_{—}035581), pig, dog and monkey.

A “functional derivative of SK1” is a protein that is derived from SK1 that still has the biological activity of SK1, i.e., to form S1P from sphingosine. Examples of functional derivative of SK1 include, but are not limited to, truncations of SK1 that contain the catalytic domain of SK1, or fusion proteins of SK1 that comprise the catalytic domain of SK1 and amino acid sequence from other protein(s).

An “SK1-activating stimulus” is any stimulus that can activate the biological activity of a SK1. Upon the activation, a SK1 catalyzes the formation of S1P from sphingosine. Various SK1-activating stimuli activate a SK1 via signal transduction involving diverse receptor families in numerous cell types. In one embodiment, SK1 is activated by proinflammatory cytokines such as TNFα via the proinflammatory cytokine receptors such as TNF-α receptor. In another embodiment, SK1 is activated by a signal conducted from a receptor tyrosine kinase, such as VEGF or PDGF receptor. In yet another embodiment, SK1 is activated by a signal conducted by high affinity FcεRI receptors. Furthermore, a SK1 is activated by a signal conducted by a GPCR, such as muscarinic receptor, S1P-receptor, or a PAR. For example, it is discovered herein that thrombin is a SK1-activating stimulus that activates SK1 via a signal transduction involving PAR1.

A “protease-activated receptor”, “PAR1”, “PAR-1”, “Par-1”, or “PAR-1 protein” each refers to a protein that is a seven transmembrane G-protein-coupled receptor that serves as the cellular receptor for thrombin in a thrombin signal transduction pathway. It is also called coagulation factor II receptor. It is activated by proteolytic cleavage.

A “PAR1”, (1) has greater than about 60% amino acid sequence identity to a human PAR1 protein (NCBI protein accession number: NP_{—}001983); (2) binds to antibodies, e.g., polyclonal or monoclonal antibodies, raised against a human PAR1 (NCBI protein accession number: NP_{—}001983); or (3) is encoded by a polynucleotide that specifically hybridizes under stringent hybridization conditions to a nucleic acid molecule having a sequence that has greater than about 60% nucleotide sequence identity to the coding region of a human PAR 1 cDNA (NCBI nucleotide accession number: NM_{—}001992).

In some embodiments, the “PAR1” has greater than about 65, 70, 75, 80, 85, 90, or 95 percent amino acid sequence identity to a human PAR1 (NCBI protein accession number: NP_{—}001983). Exemplary PAR1 includes human PAR1, which includes structural and functional polymorphisms of the human PAR1 protein depicted in NCBI protein accession number: NP_{—}001983. Par1 also includes orthologs of the human PAR1 in other animals such as rat (i.e., NCBI nucleotide accession NO: NP_{—}037082), mouse (i.e., NCBI protein accession NO: NP_{—}034299), pig, dog and monkey.

A “signal transduction” is the cascade of processes by which an extracellular signal interacts with a receptor at a cell surface, causing a change in the level of a second messenger, and ultimately effects a change in the cell function.

A “thrombin signal transduction” refers to a signal transduction, wherein the extracellular signal is thrombin. In one embodiment, a “thrombin signal transduction” is the cascade of processes by which thrombin binds to a PAR-1, -3 or -4 receptor at a cell surface, causing a change in the level of a second messenger, such as calcium, cyclic AMP, or S1P, and ultimately effects a change in the cell's function. The change in the cell's function can be the change of any cellular process thrombin is involved in. For example, thrombin signal transduction can result in changes in coagulation, cellular responses to tissue injury, the expression of adhesion molecules, vascular permeability, angiogenesis, and the release of cytokines and growth factors, etc.

“Nucleic acid sequence” or “nucleotide sequence” refers to the arrangement of either deoxyribonucleotide or ribonucleotide residues in a polymer in either single- or double-stranded form. Nucleic acid sequences can be composed of natural nucleotides of the following bases: thymidine, adenine, cytosine, guanine, and uracil; abbreviated T, A, C, G, and U, respectively, and/or synthetic analogs.

The term “oligonucleotide” refers to a single-stranded DNA or RNA sequence of a relatively short length, for example, less than 100 residues long. For many methods, oligonucleotides of about 16-25 nucleotides in length are useful, although longer oligonucleotides of greater than about 25 nucleotides may sometimes be utilized. Some oligonucleotides can be used as “primers” for the synthesis of complimentary nucleic acid strands. For example, DNA primers can hybridize to a complimentary nucleic acid sequence to prime the synthesis of a complimentary DNA strand in reactions using DNA polymerases. Oligonucleotides are also useful for hybridization in several methods of nucleic acid detection, for example, in Northern blotting or in situ hybridization.

A “polypeptide sequence” or “protein sequence” refers to the arrangement of amino acid residues in a polymer. Polypeptide sequences can be composed of the standard 20 naturally occurring amino acids, in addition to rare amino acids and synthetic amino acid analogs. Shorter polypeptides are generally referred to as peptides.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals that are involved in the synthesis of the protein. Accordingly such preparations of the protein have less than about 30%, 20%, 10%, 5% (by dry weight) of chemical precursors or compounds other than the polypeptide of interest.

Isolated biologically active polypeptide can have several different physical forms. The isolated polypeptide can exist as a full-length nascent or unprocessed polypeptide, or as a partially processed polypeptide or as a combination of processed polypeptides. The full-length nascent polypeptide can be postranslationally modified by specific proteolytic cleavage events that result in the formation of fragments of the full-length nascent polypeptide. A fragment, or physical association of fragments can have the biological activity associated with the full-length polypeptide; however, the degree of biological activity associated with individual fragments can vary. An isolated or substantially purified polypeptide, can be a polypeptide encoded by an isolated nucleic acid sequence, as well as a polypeptide synthesized by, for example, chemical synthetic methods, and a polypeptide separated from biological materials, and then purified, using conventional protein analytical or preparatory procedures, to an extent that permits it to be used according to the methods described herein.

“Recombinant” refers to a nucleic acid, a protein encoded by a nucleic acid, a cell, or a viral particle, that has been modified using molecular biology techniques to something other than its natural state. For example, recombinant cells can contain nucleotide sequence that is not found within the native (non-recombinant) form of the cell or can express native genes that are otherwise abnormally expressed, under-expressed, or not expressed at all. Recombinant cells can also contain genes found in the native form of the cell wherein the genes are modified and re-introduced into the cell by artificial means. The term also encompasses cells that contain an endogenous nucleic acid that has been modified without removing the nucleic acid from the cell; such modifications include those obtained, for example, by gene replacement, and site-specific mutation.

A “recombinant host cell” or “recombinant cell” is a cell that has had introduced into it a recombinant DNA sequence. Recombinant DNA sequence can be introduced into host cells using any suitable method including, for example, electroporation, calcium phosphate precipitation, microinjection, transformation, biolistics and viral infection. Recombinant DNA may or may not be integrated (covalently linked) into chromosomal DNA making up the genome of the cell. For example, the recombinant DNA can be maintained on an episomal element, such as a plasmid. Alternatively, with respect to a stably transformed or transfected cell, the recombinant DNA has become integrated into the chromosome so that it is inherited by daughter cells through chromosome replication. This stability is demonstrated by the ability of the stably transformed or transfected cell to establish cell lines or clones comprised of a population of daughter cells containing the exogenous DNA. Recombinant host cells may be prokaryotic or eukaryotic, including bacteria such as E. coli, fungal cells such as yeast, mammalian cells such as cell lines of human, bovine, porcine, monkey and rodent origin, and insect cells such as Drosophila- and silkworm-derived cell lines. It is further understood that the term “recombinant host cell” refers not only to the particular subject cell, but also to the progeny or potential progeny of such a cell. Because certain modifications can occur in succeeding generations due to either mutation or environmental influences, such progeny may not, in fact, be identical to the parent cell, but are still included within the scope of the term as used herein.

As used herein, “operably linked”, refers to a functional relationship between two nucleic acid sequences. For example, a promoter sequence that controls expression (for example, transcription) of a coding sequence is operably linked to that coding sequence. Operably linked nucleic acid sequences can be contiguous, typical of many promoter sequences, or non-contiguous, in the case of, for example, nucleic acid sequences that encode repressor proteins. Within a recombinant expression vector, “operably linked” is intended to mean that the coding sequence of interest is linked to the regulatory sequence(s) in a manner that allows for expression of the coding sequence, e.g., in an in vitro transcription/translation system or in a host cell when the vector is introduced into the host cell.

“Vector” or “construct” refers to a nucleic acid molecule into which a heterologous nucleic acid can be or is inserted. Some vectors can be introduced into a host cell allowing for replication of the vector or for expression of a protein that is encoded by the vector or construct. Vectors typically have selectable markers, for example, genes that encode proteins allowing for drug resistance, origins of replication sequences, and multiple cloning sites that allow for insertion of a heterologous sequence. Vectors are typically plasmid-based and are designated by a lower case “p” followed by a combination of letters and/or numbers. Starting plasmids disclosed herein are either commercially available, publicly available on an unrestricted basis, or can be constructed from available plasmids by application of procedures known in the art. Many plasmids and other cloning and expression vectors that can be used in accordance with the present invention are well-known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

“Sequence” means the linear order in which monomers occur in a polymer, for example, the order of amino acids in a polypeptide or the order of nucleotides in a polynucleotide.

“Sequence identity or similarity”, as known in the art, is the relationship between two or more polypeptide sequences or two or more polynucleotide sequences, as determined by comparing the sequences. As used herein, “identity”, in the context of the relationship between two or more nucleic acid sequences or two or more polypeptide sequences, refers to the percentage of nucleotide or amino acid residues, respectively, that are the same when the sequences are optimally aligned and analyzed. For purposes of comparing a queried sequence against, for example, the amino acid sequence of human SK1 (NCBI protein accession number: NP_{—}068807), the queried sequence is optimally aligned with human SK1 and the best local alignment over the entire length of human SK1 is obtained.

Analysis can be carried out manually or using sequence comparison algorithms. For sequence comparison, typically one sequence acts as a reference sequence, to which a queried sequence is compared. When using a sequence comparison algorithm, test and reference sequences are input into a computer, sub-sequence coordinates are designated, if necessary, and sequence algorithm program parameters are designated.

Optimal alignment of sequences for comparison can be conducted, for example, by using the homology alignment algorithm of Needleman & Wunsch, J Mol. Biol., 48:443 (1970). Software for performing Needleman & Wunsch analyses is publicly available through the Institut Pasteur (France) Biological Software website: http://bioweb.pasteur.fr/seqanal/interfaces/needle.html. The NEEDLE program uses the Needleman-Wunsch global alignment algorithm to find the optimum alignment (including gaps) of two sequences when considering their entire length. The identity is calculated along with the percentage of identical matches between the two sequences over the reported aligned region, including any gaps in the length. Similarity scores are also provided wherein the similarity is calculated as the percentage of matches between the two sequences over the reported aligned region, including any gaps in the length. Standard comparisons utilize the EBLOSUM62 matrix for protein sequences and the EDNAFULL matrix for nucleotide sequences. The gap open penalty is the score taken away when a gap is created; the default setting using the gap open penalty is 10.0. For gap extension, a penalty is added to the standard gap penalty for each base or residue in the gap; the default setting is 0.5.

Hybridization can also be used as a test to indicate that two polynucleotides are substantially identical to each other. Polynucleotides that share a high degree of identity will hybridize to each other under stringent hybridization conditions. “Stringent hybridization conditions” has the meaning known in the art, as described in Sambrook et al., Molecular Cloning: A Laboratory Manual, Second Edition, Cold Spring Harbor Laboratory, Cold Spring Harbor, N.Y., (1989). An exemplary stringent hybridization condition comprises hybridization in 6× sodium chloride/sodium citrate (SSC) at about 45° C., followed by one or more washes in 0.2×SSC and 0.1% SDS at 50-65° C., depending upon the length over which the hybridizing polynucleotides share complementarity.

A “reporter gene” refers to a nucleic acid sequence that encodes a reporter gene product. As is known in the art, reporter gene products are typically easily detectable by standard methods. Exemplary suitable reporter genes include, but are not limited to, genes encoding luciferase (lux), β-galactosidase (lacZ), green fluorescent protein (GFP), chloramphenicol acetyltransferase (CAT), β-glucuronidase, neomycin phosphotransferase, and guanine xanthine phosphoribosyl-transferase proteins.

Methods of Evaluating the Effectiveness of a Treatment Involving a Compound that Increases or Decreases SK1 Activity

In one general aspect, the invention provides a method of determining a biological activity of a SK1 in a cell. Such a method comprises the step of determining the expression level of a MCP-1 gene from the cell.

In another general aspect, the present invention provides a method of monitoring the effect of a compound administered to a subject, wherein said compound is expected to increase or decrease the biological activity of a sphingosine kinase-1 in a cell of said subject. Such a method comprises the step of measuring the expression level of a monocyte chemoattractant protein-1 gene from the cell of said subject. The compound can be administered to the subject for the treatment or prevention of various pathological conditions such as cardiovascular diseases, atherosclerosis, diabetes, stroke, autoimmune and inflammatory diseases, allergic diseases such as dermatitis, T helper-1 related diseases, chronic obstructive pulmonary disease, asthma, cell proliferative diseases such as cancer, neurodegenerative disorders, or thrombosis.

A biological sample taken from a subject can be used to determine the expression level of a MCP-1 gene in a cell in the subject. Any suitable methods known to a skilled artisan can be used to obtain the biological sample. For example, the biological sample can be obtained from epithelia where MCP-1 is mostly expressed, i.e., via needle biopsy. The biological sample can also be obtained from blood or plasma.

In some embodiments, the expression level of a MCP-1 gene in a cell can be determined by measuring the mRNA amount of the gene in the cell. The amount of mRNA of a particular gene in a biological sample can be measured using a number of techniques. For example, mRNA can be measured by contacting the biological sample with a compound or an agent capable of specifically detecting the mRNA. Often a labeled nucleic acid probe capable of hybridizing specifically to the mRNA is used. For example, the nucleic acid probe specific for human MCP1 mRNA can be a full-length human MCP-1 cDNA (NCBI nucleotide accession number: NM_{—}002982), or a portion thereof, such as an oligonucleotide of at least 15, 30, 50, 100, 250 or 500 nucleotides in length that can hybridize to human MCP-1 mRNA under stringent hybridization conditions. Under stringent conditions, the nucleic acid probe specific for human MCP-1 mRNA will only hybridize to this mRNA but not the other mRNA species present in the testing biological sample. Useful nucleic acid probes for the invention include those capable of hybridizing to a human MCP-1 cDNA (NCBI nucleotide accession number: NM_{—}002982) under stringent hybridization conditions.

Another technique for determining the mRNA amount of a particular gene in a biological sample is quantitative real-time reverse transcription polymerase chain reaction (RT-PCR). Complementary DNA (cDNA) of a gene, for example a human MCP-1 gene, can be prepared from the sample via reverse transcription. The cDNA can be amplified via PCR using oligonucleotide primers capable of hybridizing to the MCP-1 cDNA under stringent hybridization conditions. Kits are commercially available that facilitate the RT-PCR, for example, the “One-Step RT-PCR Master Mix Reagent” kit from Applied Biosystems (Foster City, Calif.).

Over the decades, in situ hybridization has been used extensively to study the distribution and expression of mRNA species of particular genes within specific compartments of a cell or tissue. Types of nucleic acid probes used for in situ hybridization assay include single-stranded oligonucleotides, single-stranded RNA probes (riboprobes), or double-stranded cDNA sequences, of various lengths. Probes can be designed specifically against any known expressed nucleic acid sequence. A number of different radioisotope and non-isotopic labels are commercially available that may be used in in-situ hybridization. For a review of in-situ hybridization methods, see McNicol et al. (1997), J. Pathol 182(3): 250-61. Other useful techniques for determining the mRNA amount of a particular gene in a biological sample include DNA microarray analysis, dot-blotting, and Northern hybridizations.

In some embodiments, the expression level of a MCP-1 gene in a biological sample can be determined by measuring the amount of polypeptide encoded by the gene. A cell expresses the MCP-1 protein from a MCP-1 gene and subsequently secrets the MCP-1 protein outside out of the cell. Therefore, in a particular embodiment of the invention, the expression level of a MCP-1 gene from a cell of said subject is measured as the amount of MCP-1 in the blood or plasma sample of the subject.

The amount of a protein in a biological sample can be measured by contacting the biological sample with a compound or an agent capable of detecting the protein specifically. For example, a preferred agent for detecting a MCP-1 protein is an antibody capable of binding specifically to a portion of the polypeptide. In one preferred method, an antibody specific for a MCP-1 protein coupled to a detectable label is used for the detection of the MCP-1 protein. Antibodies can be polyclonal or monoclonal. A whole antibody molecule or a fragment thereof (e.g., Fab or F(ab′)₂) can be used. Antibodies are available through specialist laboratories. For example, antibodies directed against synthetic peptide sequences specific to MCP-1 protein can be developed within a relatively short time scale, enabling a greater degree of flexibility for studying these targets of interest.

Techniques for detection of a polypeptide such as the MCP-1 protein include enzyme linked immunosorbent assays (ELISAs), Western blots, immunoprecipitations and immunofluorescence, and immunohistochemistry. Details for performing these methods can be found in, for example, Sambrook et al. Molecular Cloning: A Laboratory Manual, Second Edition, Cold Spring Harbor Laboratory, Cold Spring Harbor, N.Y., (1989)).

In addition, the expression level of a MCP-1 gene in a living human organ can be determined by quantitative noninvasive means, such as positron emission tomography (PET) imaging (Sedvall et al., (1988) Psychopharmacol. Ser., 5:27-33). For example, trace amounts of the Pa9 protein binding radiotracers can be injected intravenously into a subject, and the distribution of radiolabeling in brown adipose tissue, liver, heart, kidney, muscle, or other organs of the subject can be imaged. Procedures for PET imaging as well as other quantitative noninvasive imaging means are known to those skilled in the art (see review by Passchier et al., (2002) Methods 27:278).

Kits for Determining the Effectiveness of a Treatment Involving a Compound that Increases or Decreases SK1 Activity

Complete assay kits can be made available in which all reagents necessary for the detection of the expression level of a MCP-1 gene are included, usually with an optimized protocol. Thus, the invention also features a kit for monitoring the effect of a compound administered to a subject, wherein said compound is expected to increase or decrease the biological activity of a sphingosine kinase-1 in a cell of said subject. Such a kit preferably comprises a compartmentalized carrier suitable to hold in close confinement at least one container. The carrier further comprises reagents capable of detecting the MCP-1 polypeptide or MCP-1 mRNA in a biological sample and means for determining the amount of the polypeptide or mRNA in the sample. The kit can also contain a control sample or a series of control samples that can be assayed and compared to the test sample contained. Each component of the kit can be enclosed within an individual container and all of the various containers are within a single package along with the instructions for determining whether a treatment involving a compound that increases or decreases MCP-1 activity in a subject is effective or not.

For an antibody-based kit, the kit can comprise, for example: (1) a first antibody (e.g., an antibody attached to a solid support) which binds to a MCP-1; and, optionally; (2) a second, different antibody which binds to either the MCP-1 or the first antibody and is conjugated to a detectable agent; (3) a substantially purified MCP-1 as positive control; and (4) an instruction for correlating the amount of MCP-1 measured from a biological sample with the effectiveness of the evaluating compound. For example, the antibody-based kit can comprise an antibody that binds specifically to a MCP-1 from human MCP-1 (NCBI protein accession number: NP_{—}002973), rat (i.e., NCBI nucleotide accession NO: NP_{—}113718), or mouse (i.e., NCBI protein accession NO: NP_{—}035463), pig, dog and monkey. The antibody can be polyclonal or monoclonal. Any suitable methods known to a skilled artisan can be used to develop the antibody.

For an oligonucleotide-based kit, the kit can comprise, for example, an oligonucleotide, e.g., a labeled oligonucleotide, which hybridizes to the mRNA of a MCP-1 gene under stringent hybridization conditions and an instruction for correlating the amount of MCP-1 gene expression measured from a biological sample with the effectiveness of the evaluating compound. For example, the kit can comprise a labeled oligonucleotide that hybridizes to a human MCP-1 cDNA (NCBI nucleotide accession number: NM_{—}002982), or complements thereof under stringent hybridization conditions. Alternatively, the kit can comprise a pair of primers useful for reverse transcription and amplification of a nucleic acid molecule from the mRNA of a MCP-1 gene. For example, the kit can comprise a pair of primers useful for amplifying a nucleic acid molecule from the mRNA of a MCP-1 gene from human or other animals such as rat, mouse, pig, dog and monkey.

Methods of Identifying Compounds that Increases or Decreases the Biological Activity of a SK1

The identification of MCP-1 gene as the target gene for SK1, also allows for the development of new screening methods or assays for identifying compounds that increases or decreases the biological activity of a SK1. Thus, another general aspect of the invention relates to methods of identifying a compound that increases or decreases the biological activity of a sphingosine kinase-1. Such methods involve the identification of compounds that alter the gene expression level of a MCP-1 gene.

The compound identification methods can be performed using conventional laboratory formats or in assays adapted for high throughput. The term “high throughput” refers to an assay design that allows easy screening of multiple samples simultaneously, and can include the capacity for robotic manipulation. Another desired feature of high throughput assays is an assay design that is optimized to reduce reagent usage, or minimize the number of manipulations in order to achieve the analysis desired. Examples of assay formats include 96-well or 384-well plates, levitating droplets, and “lab on a chip” microchannel chips used for liquid-handling experiments. As known by those in the art, as miniaturization of plastic molds and liquid-handling devices are advanced, or as improved assay devices are designed, greater numbers of samples will be able to be screened more efficiently using the inventive assay.

Candidate compounds for screening can be selected from numerous chemical classes, preferably from classes of organic compounds. Although candidate compounds can be macromolecules, preferably the candidate compounds are small-molecule organic compounds, i.e., those having a molecular weight of greater than 50 and less than 2500. Candidate compounds have one or more functional chemical groups necessary for structural interactions with polypeptides. Preferred candidate compounds have at least an amine, carbonyl, hydroxyl or carboxyl group, preferably at least two such functional groups, and more preferably at least three such functional groups. The candidate compounds can comprise cyclic carbon or heterocyclic structural moieties and/or aromatic or polyaromatic structural moieties substituted with one or more of the above-exemplified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound is preferably a DNA or RNA molecule, although modified nucleic acids having non-natural bonds or subunits are also contemplated.

Candidate compounds may be obtained from a variety of sources, including libraries of synthetic or natural compounds. For example, numerous means are available for random and directed synthesis of a variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid-phase or solution-phase libraries: synthetic library methods requiring deconvolution; the “one-bead one-compound” library method; and synthetic library methods using affinity chromatography selection (see, e.g., Lam (1997), Anticancer Drug Des. 12:145). Alternatively, libraries of natural compounds in the form of bacterial, fungal, plant and animal extracts are available or may be routinely produced. Additionally, natural and synthetically produced libraries and compounds can be routinely modified through conventional chemical, physical, and biochemical means.

Further, known pharmacological agents can be subjected to directed or random chemical modifications, such as acylation, alkylation, esterification, acidification, etc., to produce structural analogs of the agents. Candidate compounds can be selected randomly or can be based on existing compounds that bind to and/or modulate the biological activity of a SK1. For example, a source of candidate agents can be libraries of molecules based on known activators or inhibitors for SK1, in which the structure of the compound is changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions.

A variety of other reagents also can be included in the mixture. These include reagents such as salts, buffers, neutral proteins (e.g., albumin), and detergents that can be used to facilitate optimal protein-protein and/or protein-nucleic acid binding. Such a reagent can also reduce non-specific or background interactions of the reaction components. Other reagents that improve the efficiency of the assay, such as nuclease inhibitors, antimicrobial agents, and the like, can also be used.

Examples of methods for the synthesis of molecular libraries can be found in the art, for example in: Zuckermann et al. (1994), J Med. Chem. 37:2678. Libraries of compounds can be presented in solution (e.g., Houghten (1992), Biotechniques 13:412-421), or on beads (Lam (1991), Nature 354:82-84), chips (Fodor (1993), Nature 364:555-556), bacteria (U.S. Pat. No. 5,223,409), spores (U.S. Pat. No. 5,571,698), plasmids (Cull et al. (1992), Proc. Natl. Acad. Sci. USA 89:1865-1869) or phage (see e.g., Scott and Smith (1990), Science 249:386-390).

In one embodiment, the invention provides a method of identifying a compound that increases or decreases the biological activity of a SK1, comprising the steps of:
a) contacting a SK1-responsive system with a solution comprising a buffer and a test compound, wherein the SK1-responsive system comprises a SK1 or a functional derivative thereof, and a gene whose expression is controlled by a regulatory sequence of a MCP-1 gene;
b) measuring from the SK1-responsive system the expression level of the gene whose expression is controlled by a regulatory sequence of a MCP-1 gene; and
c) identifying the compound by its ability to increase or decrease said expression level as compared to a control wherein the SK1-responsive system is contacted with only the buffer.

A “SK1-responsive system” is used in its broadest sense and refers to single cells, tissues, and complex multicellular organisms such as mammals, that are responsive to stimulation of SK1, for example by TNFα. In one embodiment, the SK1-responsive system is an animal, a tissue, or a cell that is a natural host for an endogenous SK1 and an endogenous MCP-1 gene. For example, endothelial cells such as HMVECs, HUVECs or HAECs are all natural SK1-responsive system that can be used in the invention. The SK1-responsive system can also be a recombinant host cell for SK1. Any suitable method known to a skilled artisan may be used to obtain such a SK1 responsive system with a recombinant SK1. For example, the SK1-responsive system can be constructed by introducing an exogenous DNA encoding a functional SK1 into a natural host cell for a MCP-1 gene. The expression level of the MCP-1 gene from such a SK1-responsive system can be measured either by the amount of mRNA or protein of the MCP-1 gene from the SK1-responsive system using methods described supra.

In another embodiment, the SK1-responsive system comprises a functional SK1 protein and a reporter gene controlled by a regulatory sequence of a MCP-1 gene. The reporter gene comprises a regulatory sequence of a MCP-1 gene and an operably linked coding sequence for a reporter. Such a system allows for transcriptional regulation of the reporter gene in response to a SK1 modulator. Therefore, the biological activity of SK1 can be measured indirectly via a reporter activity. For example, when a luciferase (luc) gene is used as the reporter gene, the biological activity of SK1 can be measured as the amount of bioluminescence from the SK1-responsive system. Other reporter genes, include, but are not limited to, genes encoding for green fluorescent protein (GFP), β-galactosidase (lacZ), chloramphenicol acetyltransferase (cat), β-glucuronidase, neomycin phosphotransferase, and guanine xanthine phosphoribosyl-transferase. The biological activity of the reporter can be easily measured. Kits are available commercially to facilitate the measurement of the reporter activity.

Any suitable methods known to a skilled artisan may be used to construct a nucleic acid comprising a coding sequence of a reporter operably linked to a regulatory sequence of MCP-1 gene. The regulatory sequence of a MCP-1 gene includes any nucleotide sequence that is naturally associated with and controls the gene expression of the MCP-1 gene. Preferably, the regulatory sequence comprises the binding sites for transcription factors NF-kB and AP-1.

In a preferred embodiment, when a compound that decreases SK1 biological activity is sought after, the method of the present invention further comprises a step of contacting the SK1-responsive system with a SK1-activating stimulus, before the step of measuring gene expression from the system. The SK1-activating stimulus can be contacted with the SK1-responsive system either before, simultaneously, or after the system is contacted with the test compound.

For example, a compound that decreases the biological activity of SK1 can be identified using a method comprising the steps of:
a) contacting an endothelial cell with a test compound;
b) contacting the endothelial cell with a SK1-activating stimulus (such as TNFα or thrombin);
c) measuring expression level of MCP-1 from the endothelial cell; and
d) identifying the compound by its ability to decrease MCP-1 gene expression as compared to a control.

Wherein step a) can be performed prior to, after, or simultaneously with step b).

In a particular embodiment, the method of the present invention further comprises the steps of confirming a candidate compound identified from step c) of the compound identification method supra in a functional assay with SK1. Such a functional assay comprises the steps of:
a) contacting a SK1 with a solution comprising the candidate compound and a buffer comprising sphingosine and ATP;
b) measuring the amount of sphingosine-1-phosphate produced from the sphingosine; and
c) confirming the candidate compound by its ability to increase or decrease the production of sphingosine-1-phosphate from the sphingosine as compared to a control wherein the SK1 is contacted with only the buffer.

A host cell (recombinant or native) that expresses a SK1 gene can be used for the functional assay. Preferably, a substantially purified SK1 or a functional derivative thereof can be used for the functional assay.

Methods of Regulating MCP-1 Gene Expression

The identification of MCP1 as a target gene for SK1 allows for the development of a new method for regulating expression of a MCP-1 gene in a cell. Regulation of MCP-1 gene expression in a cell directly regulates the amount of MCP-1 produced from the cell, ultimately effects a change in the cell functioning involving MCP-1. Thus, another general aspect of the invention relates to methods of increasing or decreasing expression of a MCP-1 gene in a cell. Such methods comprise the step of increasing or decreasing the biological activity of a sphingosine-1-phosphate in the cell such that expression of said monocyte chemoattractant protein-1 gene is increased or decreased, respectively.

It has been shown that absence of MCP-1 expression provides sustained protection from atherosclerosis lesion development in several atherosclerosis models (Gosling et al., J. Clin Invest, 1999, 103:773-778; and Gu et al., Mol Cell, 1998, 2:275-281). Thus, the present invention provides a method of treating or preventing atherosclerosis in a subject, comprising a step of decreasing the biological activity of a sphingosine-1-phosphate in a cell of the subject, such that atherosclerosis in the subject is treated or prevented. Similar method can be used to treat or prevent other diseases or disorders that can be treated or prevented by regulating expression of MCP1 gene.

In one embodiment, the methods comprise the step of administering to the cell a compound that increases or decreases the biological activity of SK1, i.e., a compound that increases or decreases the ability of SK1 to catalyze the formation of S1P from sphingosine. Examples of such a compound include dimethylsphingosine (DMS). Such compounds can also be identified using the methods of compound identification described supra.

In another embodiment, the methods comprise the step of increasing or decreasing expression of a SK1 gene in the cell. In one embodiment, antisense can be used to decrease the expression of a SK1 gene in a cell when decreased expression or biological activity of SK1 is desirable.

The principle of antisense-based strategies is based on the hypothesis that sequence-specific suppression of gene expression can be achieved by intracellular hybridization between mRNA and a complementary antisense species. The formation of a hybrid RNA duplex can then interfere with the processing/transport/translation and/or stability of the target mRNA, such as that of the SK1 gene. Hybridization is required for the antisense effect to occur. Antisense strategies can use a variety of approaches including the use of antisense oligonucleotides, injection of antisense RNA and transfection of antisense RNA expression vectors. Phenotypic effects induced by antisense hybridization to a sense strand are based on changes in criteria such as protein levels, protein activity measurement, and target mRNA levels.

An antisense nucleic acid can be complementary to an entire coding strand of a target gene, or to only a portion thereof. An antisense nucleic acid molecule can also be complementary to all or part of a non-coding region of the coding strand of a target gene. The non-coding regions (“5′ and 3′ UTRs”) are the 5′ and 3′ sequences which flank the coding region and are not translated into amino acids. Preferably, the non-coding region is a regulatory region for the transcription or translation of the target gene.

An antisense oligonucleotide can be, for example, about 15, 25, 35, 45 or 65 nucleotides or more in length taken from the complementary sequence of a SK1 cDNA. It is preferred that the sequence be at least 18 nucleotides in length in order to achieve sufficiently strong annealing to the target mRNA sequence to prevent translation of the sequence. (Izant et al., 1984, Cell, 36:1007-1015; Rosenberg et al., 1985, Nature, 313:703-706). An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxytnethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylecytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. An antisense nucleic acid molecule can be a CC-anomeric nucleic acid molecule. A CC-anomeric nucleic acid molecule forms specific double-stranded hybrids with complementary RNA in which, contrary to the usual P-units, the strands run parallel to each other (Gaultier et al. (1987) Nucleic Acids Res. 15:6625-664 1). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

Alternatively, the antisense nucleic acid can also be produced biologically using an expression vector into which a nucleic acid has been subcloned in an antisense orientation as described supra. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. To achieve sufficient intracellular concentrations of the antisense molecules, vector constructs in which the antisense nucleic acid molecule is placed under the control of a strong pol II or pol III promoter are preferred. For a discussion of the regulation of gene expression using antisense genes see Weintraub et al. (1985, Trends in Genetics, Vol. 1(1), pp. 22-25).

Typically, antisense nucleic acid is administered to a subject by microinjection, liposome encapsulation or generated in situ by expression from vectors harboring the antisense sequence. An example of a route of administration of antisense nucleic acid molecules includes direct injection at a tissue site. The antisense nucleic acid can be ligated into viral vectors that mediate transfer of the antisense nucleic acid when the viral vectors are introduced into host cells. Suitable viral vectors include retrovirus, adenovirus, adeno-associated virus, herpes virus, vaccinia virus, polio virus and the like. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens.

Once inside the cell, antisense nucleic acid molecules hybridize with or bind to cellular mRNA and/or genomic DNA encoding a SK1 protein to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix.

In a preferred embodiment, the method involves the use of small interfering RNA (siRNA). Many organisms possess mechanisms to silence gene expression when double-stranded RNA (dsRNA) corresponding to the gene is present in the cell through a process known as RNA interference. The technique of using dsRNA to reduce the activity of a specific gene was first developed using the worm C. elegans and has been termed RNA interference, or RNAi (Fire, et al., (1998), Nature 391: 806-811). RNAi has since been found to be useful in many organisms, and recently has been extended to mammalian cells (see review by Moss, (2001), Curr Biol 11: R772-5). An important advance was made when RNAi was shown to involve the generation of small RNAs of 21-25 nucleotides (Hammond et al., (2000) Nature 404: 293-6; Zamore et al., (2000) Cell 101: 25-33). These small interfering RNAs, or siRNAs, may initially be derived from a larger dsRNA that begins the process, and are complementary to the target RNA that is eventually degraded. The siRNAs are themselves double-stranded with short overhangs at each end. They act as guide RNAs, directing a single cleavage of the target in the region of complementarity (Elbashir et al., (2001) Genes Dev 15: 188-200; Zamore et al., (2000) Cell 101: 25-33).

siRNAs comprising about 21-25 nucleotides complementary to nucleotide sequence shown in SEQ ID NO: 1, 3, or 5 can be used in the method of treatment. Methods of producing siRNA are known to those skilled in the art. For example, WO0175164 A2 described methods of producing siRNA of 21-23 nucleotides (nt) in length from an in vitro system, and using such siRNA to interfere with mRNA of a gene in a cell or organism. The siRNA can also be made in vivo from a mammalian cell using a stable expression system. For example, a vector system, named pSUPER, that directs the synthesis of siRNAs in mammalian cells, was recently reported (Brummelkamp et al., (2002) Science 296: 550-3). An example of using siRNA to reduce gene expression in a cell is shown in Example 3.

In a particular embodiment, the present invention provides a method of decreasing the expression in an endothelial cell of a monocyte chemoattractant protein-1 gene, comprising the step of:
(a) introducing into the endothelial cell siRNA that targets the mRNA of a SK1 gene for degradation;
(b) maintaining the cell produced in (a) under conditions under which siRNA interference of the mRNA of the SK1 gene in the cell occurs.
   The siRNA can be introduced into a cell using procedures similarly to those for the anti-sense nucleic acids described herein.

In another embodiment, the method comprising the step of introducing a nucleic acid molecule capable of expressing a SK1 gene into a cell, when increased expression of MCP-1 gene in the cell is desired.

As one example, a DNA molecule encoding a SK1 gene can be first cloned into a retroviral vector. The expression of the target gene from the vector can be driven from its endogenous promoter or from the retroviral long terminal repeat or from a promoter specific for certain target cells. The vector can then be introduced into a cell to successfully express the target gene in the cell. The gene can be preferably delivered to the cell in a form which can be used by the cell to encode sufficient protein to provide effective function. Retroviral vectors are often a preferred gene delivery vector because of their high efficiency of infection and stable integration and expression. Alternatively, the DNA molecule encoding a target gene can be transferred into cells by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo gene therapy. Protocols for molecular methodology of gene therapy suitable for use with the methods of the invention are described in Gene Therapy Protocols, edited by Paul D. Robbins, Human press, Totowa N.J., 1996.

A procedure for performing ex vivo gene therapy is outlined in U.S. Pat. No. 5,399,346 and also in exhibits submitted in the file history of that patent, all of which are publicly available documents. In general, gene therapy can involve introduction in vitro of a functional copy of a gene into a cell(s) of a subject, and returning the genetically engineered cell(s) to the subject. The functional copy of the gene is under operable control of regulatory elements, which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy uses vectors such as adenovirus, retroviruses, vaccinia virus, bovine papilloma virus, and herpes virus such as Epstein-Barr virus. Gene transfer can also be achieved using non-viral means requiring infection in vitro. Such means can include calcium phosphate, DEAE dextran, electroporation, and protoplast fusion. Targeted liposomes can also be potentially beneficial for delivery of DNA into a cell.

During treatment, the effective amount of nucleic acid molecules of the invention administered to individuals can vary according to a variety of factors including type, species, age, weight, sex and medical condition of the patient; the severity of the condition to be treated; the route of administration; the renal and hepatic function of the patient; and the particular nucleic acid molecule thereof employed. A physician or veterinarian of specialized skill in gene therapy can determine and prescribe the effective amount required to prevent, counter or arrest the progress of the condition. Optimal precision in achieving concentrations within the range that yields efficacy without toxicity requires a regimen based on the kinetics of the nucleic acid molecule's availability to target sites. This involves a consideration of the distribution, equilibrium, and elimination of the nucleic acid molecule involved in gene therapy.

The gene therapy disclosed herein can be used alone at appropriate dosages defined by routine testing in order to obtain optimal increase or decrease of the MCP-1 activity while minimizing any potential toxicity. In addition, co-administration or sequential administration of other agents may be desirable. The dosages of administration are adjusted when several agents are combined to achieve desired effects. Dosages of these various agents can be independently optimized and combined to achieve a synergistic result wherein the pathology is reduced more than it would be if either agent were used alone.

Methods of Regulating Thrombin Signal Transduction

The identification of thrombin as an activating stimulus for SK1 allows for the development of a new method for regulating thrombin signal transduction in a cell, ultimately effects a change in the cell functioning involving thrombin. Thus, another general aspect of the invention relates to methods of increasing or decreasing thrombin signal transduction in a cell. Such methods comprise the step of increasing or decreasing the biological activity of sphingosine-1-phosphate in the cell such that the signal transduction is increased or decreased, respectively. The method can be used to treat or prevent diseases or disorders that are related to thrombin signal transduction.

As described supra, the biological activity of a sphingosine-1-phosphate in the cell can be regulated by using a compound that increases or decreases the catalytic activity of the SK1, or by using a nucleic acid technology such as antisense, siRNA, or expression vector, etc.

The following examples illustrate the present invention without, however, limiting the same thereto.

### EXAMPLE 1

### cDNA Microarray Studies of Gene Expression in HMVECS

In an effort to rapidly assess the potential overall role of SK1 in endothelial cell function, a cDNA microarray analyses was performed in HMVEC in the absence and presence of the SK1 inhibitor DMS. The cells were stimulated through two receptor subtypes: G-protein coupled receptors, for which the Protease Activate Receptor (PAR) family was utilized with thrombin as the agonist and cytokine receptors with TNF-α as the agonist

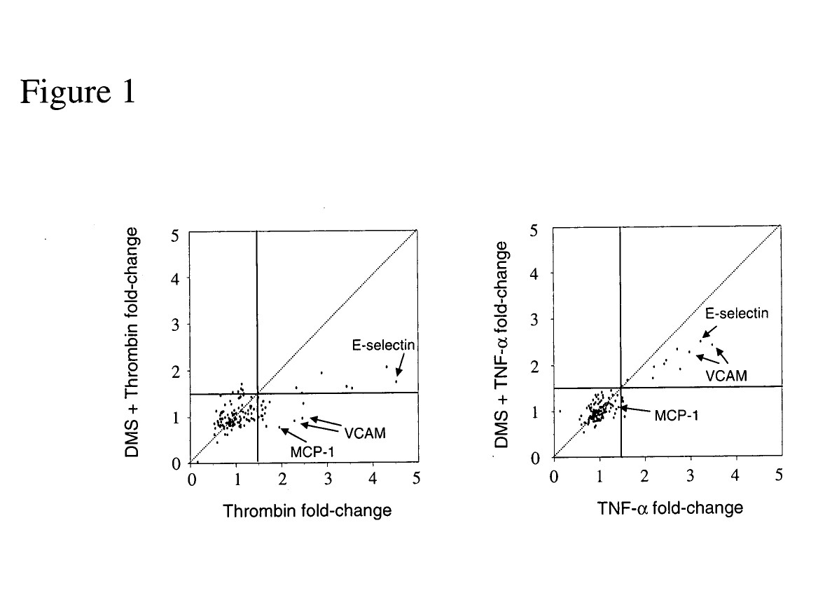

A total of 138 genes were identified that were either induced or repressed in one of the experimental conditions. Dot blots were generated to visualize the genes that were detected beyond the selected threshold value by plotting the fold-change results along the two axes from stimulated cells in the absence or presence of DMS (FIG. 1). These analyses showed that sphingosine kinase is linked to signals generated through the Thrombin receptor, a never before observed occurrence. Whereas, previous researchers had demonstrated activation of SKs through cytokine receptors, receptor tyrosine kinases, and other GPCRs, this is the first such example whereby SKs are required for selective thrombin mediated events in endothelial cells. Furthermore, previous data has shown the dependence of adhesion molecules expression, such as E-selectin or VCAM on hSK activity, thrombin stimulated HMVECs requires hSK activity for the induction of these molecules as well. Reviewing the microarray results, we chose to focus on one transcript that was induced by both receptor classes and appeared down-modulated by DMS; the inflammatory protein monocyte chemoattractant protein-1 (MCP-1). As shown in FIG. 1, MCP-1 was induced in response to both ligands, thrombin and TNF-α, and was inhibited in conditions where DMS was added prior to stimulation.

Materials—Human thrombin was purchased from American Diagnostica, Inc. (Stamford, Conn.). TNF-α was purchased from R&D Systems, dimethylsphingosine (DMS) and Sphingosine-1-phosphate were from Avanti, PAR peptides were prepared internally at J&J, MCP-1 ELISA kit (Hycult Biotechnology), Bay11-7092, GF109203, and PD98059 were from Calbiochem.

Cell Culture—Adult human microvascular endothelial cells (HMVECs) (Cambrex) were cultured in EGM complete media (Cambrex). Cells were used between the third and sixth passages for all studies.

Microarray Study—Adult human microvascular endothelial cells (HMVECs) (Cambrex) were placed into culture (5% FBS) and stimulated with thrombin (100 nM) or TNF-α (20 ng/ml) in the absence or presence of DMS (10 μM), a potent inhibitor of sphingosine kinase. After four hours of stimulation, RNA was isolated (Tri-Reagent, Bio-Mol), DNAse treated and cleaned using the RNeasy maxi kit (Qiagen). Upon validation of RNA purity using the Agilent 2100 Bioanalyzer, RNA was then subjected to cDNA microarray analyses.

A cDNA microarray containing 3563 cDNA clones was used in this study. In the gene expression studies two types of replications were used, biological and technical. Duplicate biological samples were harvested for each experimental condition for the initial microarray analyses. Additionally technical replication was employed as all samples were run in triplicate on separate microarrays. The microarray data from each sample were subjected to outlier removal based on technical replication and normalization based on both the technical and biological replication. The normalization consisted of an initial normalization between hybridization replicates within a single sample, followed by a secondary normalization across all samples within the study (Shaw et al., J Mol Microbiol Biotechnol, 2003, 5:105-122). Background hybridization levels were estimated empirically for each sample in order to assign absent and present calls to each clone within the sample. No comparisons were made between treatment and control conditions where both intensities were deemed absent. After cleanup and subsequent normalizations, a single ratio was calculated for each treatment to its assigned control. For surveying gene expression patterns genes must have had one ratio out of the 17 exhibit a fold-change ratio increase or decrease of at least 1.5. Additionally T statistics were used on the normalized data to find those genes that differed at a 0.05 significance level. Significant genes were visualized using S-PLUS 6.1 for windows (Insightful Corporation, Seattle, Wash.).

Chen et al. discloses that SK1 mediates TNFα-induced MCP-1 gene expression in endothelial cells (Chen et al., Am. J. Physiol Heart Circ physiol, 2004, 287:H1452-58).

### EXAMPLE 2

### Specific Inhibition of SK-1 Abrogates Induction of MCP-1 in HMVECS

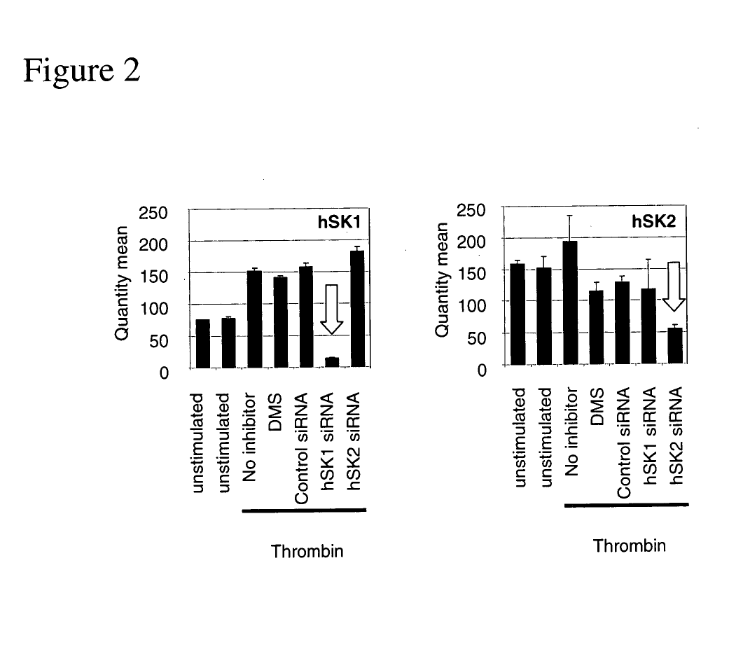

While DMS has been shown to inhibit SK activity at lower concentrations, at higher concentrations it can also affect activity of protein kinase C family members as well as casein kinases. Therefore, small interfering RNAs (siRNA) were used to selectively inhibit SK by targeting exclusively the SK family members, specifically human SK1 or SK2. With the addition of a fluorescein to the 3′ end of the siRNAs, we were able to visualize the transfection efficiency of the siRNA, which neared 100%. The siRNAs designed showed specificity for their respective human SK (hSK) isoforms as detected by Taqman quantitative RT-PCR (FIG. 2). When detecting transcripts for hSK1, it was observed that only the hSK1-specific siRNA inhibited hSK1 expression, while the hSK2 specific siRNA and the control non-silencing siRNA had no effect on hSK1 expression patterns. Similarly, when detecting transcript levels of hSK2, only the hSK2-specific siRNA affected expression of hSK2 while the hSK1-specific siRNA and the control non-silencing siRNA did not effect expression of hSK2. Therefore, the designed siRNAs showed specificity towards the intended target with no crossreactivity among hSK family members.

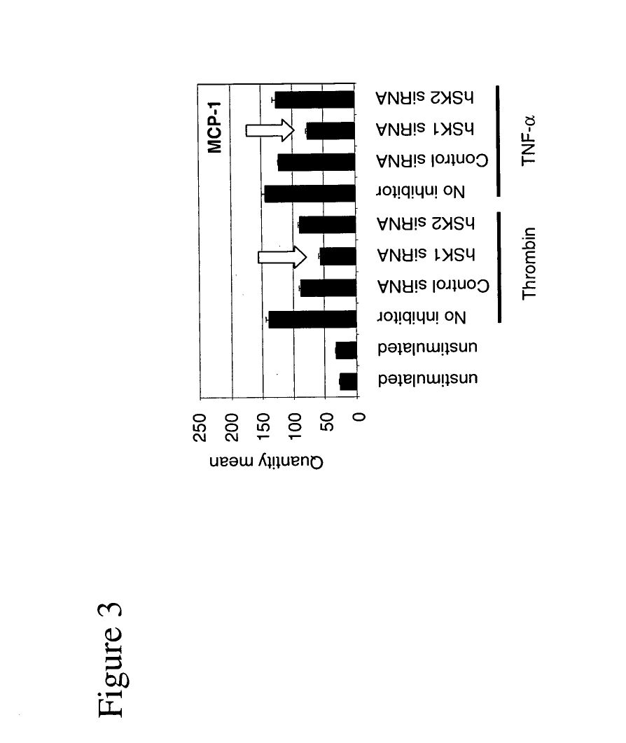

To more directly identify which hSK isoform was involved in induction of MCP-1 transcripts from HMVECs, microarray analyses were repeated employing hSK1-specific, hSK2-specific, or control non-silencing siRNAs prior to stimulation with thrombin (data not shown) and subsequently verified by quantitative RT-PCR (FIG. 3). Thrombin mediated activation of HMVECs led to an induction of MCP-1 transcripts as had been previously seen with microarray analyses (FIG. 1). In the presence of hSK1-specific siRNAs, induction of MCP-1 was inhibited whereas in the presence of hSK2-specific or control non-silencing siRNAs, MCP-1 induction was not affected.

Evaluation of siRNA Transfection Efficiency and Silencing Capabilities—Small interfering RNAs (siRNAs) were designed and synthesized to target either sequences for hSK1, SEQ ID NO: 1 (AAGAGCTGCAAGGCCTTGCCC) or hSK2, SEQ ID NO:2 (AACCTCATCCAGACAGAACGA) transcripts as well as control non-silencing siRNA, SEQ ID NO:3 (AATCTCCGAACGTGTCACGT), which has no sequence target in the human genome (Qiagen). The oligonucleotides were fluorescein-conjugated on the 3′ end of the sense strand, which facilitated visualization of transfection efficiency by fluorescence confocal microscopy (LSM 510, Zeiss). HMVECs were placed into culture (4×10³ cells/well) and transfected with 1.6 μg siRNA in a 6 well plate following manufacturer's instructions (Transmessenger, Qiagen) and 5 hours later confocal images were captured. For experimental studies RNA were isolated, DNAse treated and subjected to quantitative RT-PCR analyses to detect transcript levels of hSK1, hSK2, MCP-1 and control 18S transcripts using predesigned primers from Applied Biosystems. Alternatively, RNA from single experimental conditions was DNAse treated (Promega) and submitted for cDNA microarray analysis as described above.

Quantitative RT-PCR—HMVECs were placed into culture overnight and stimulated with thrombin (100 nM) after which RNA was isolated and DNAse treated. TaqMan® quantitative RT-PCRs were performed in triplicate as validation of microarray analyses according to manufacturers instructions (Applied Biosystems). The quantity mean for each detector was normalized to that of the 18S detector.

### EXAMPLE 3

### PAR-Expression in HMVECS is Limited to PAR-1, PAR-2 and PAR-4

Thrombin, being a serine protease, can act upon many substrates, but we were interested in the thrombin activity of PAR activation. To date there are 4 human PARs identified and we wanted to define the expression profile of PARs in HMVECs, by performing RT-PCR analyses. The RT-PCR results showed that HMVECs express the thrombin-sensitive PAR-1 and PAR-4, and the thrombin-insensitive, trypsin-sensitive PAR-2. Under our assay condition, RT-PCR failed to amplify the desired band for the third thrombin sensitive receptor, PAR-3, suggesting that HMVECs do not express or expresses at very low level of PAR-3.

RT-PCR—HMVECs were placed into culture RNA isolated (TriReagent—BioMol) and subsequently DNAse treated and cleaned using the RNeasy Maxi kit (Qiagen). RT-PCR was performed using the GC-Rich PCR reagents (Invitrogen) and SuperScript II Reverse transcriptase (Invitrogen). Results were visualized by U.V. gel electrophoresis and images captured (Polaroid).

### EXAMPLE 4

### PAR-1-Dependent MCP-1 Protein Secretion from HMVECs is Inhibited by DMS

Thrombin mediates its primary responses on cells through the receptor PAR-1. We therefore investigated whether or not pretreatment of the HMVECs with DMS inhibited the action of the PAR-1-specific activating peptide TFLLRN (PAR-1-AP) by ELISA.

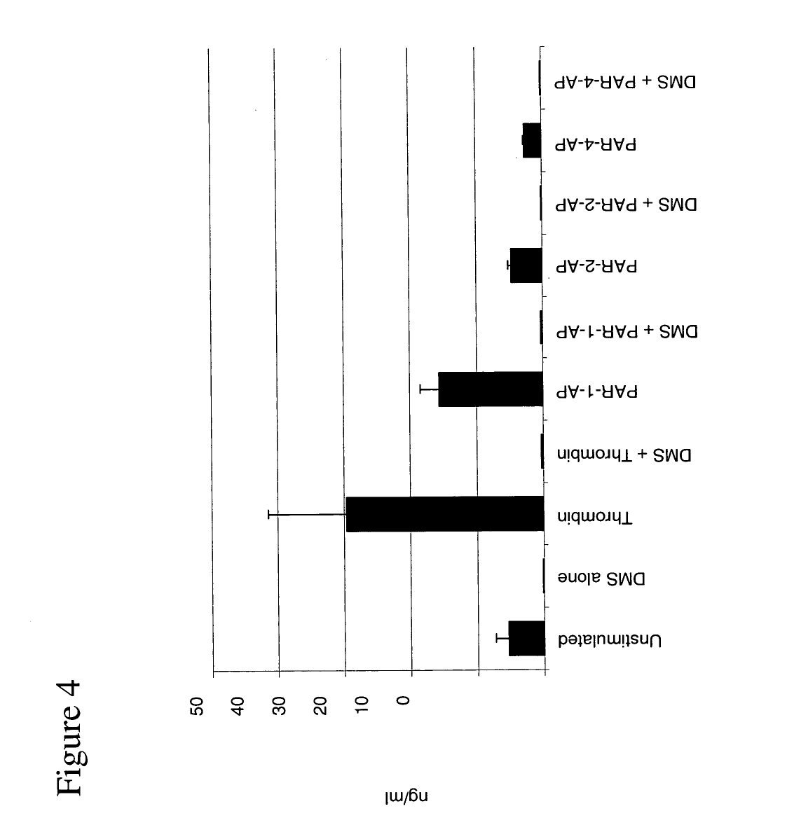

HMVECs were placed into culture and stimulated with Thrombin (100 nM) or varying concentrations of PAR-activating peptides. 24 hours later, we used ELISA to detect secretion of MCP-1 into supernatant. As shown in FIG. 4, upon stimulation of the cells with thrombin, MCP-1 protein secretion was augmented over that of unstimulated cells. Furthermore, only the PAR-1-AP stimulated secretion of MCP-1 from HMVECs, whereas neither the PAR-2-specific activating peptide (PAR-2-AP: SLIGRL) nor the PAR-4-specific activating peptides (PAR-4-AP: AYPGKF) modulated secreted MCP-1 levels as detected in the conditioned media. As had previously been demonstrated using MCP-1 transcript levels, pretreatment of HMVECs with DMS prior to stimulation with thrombin or PAR-1 agonist peptide inhibited secretion of MCP-1 protein.

MCP-1 ELISA—HMVECs were placed into culture in 96-well dishes (2×10³ cells/well) and grown for 24 hours in complete growth media. Cells were transfected with control non-silencing siRNA or siRNAs for hSK1 or hSK2 in serum-free Opti-MEM (Gibco) for 4 hours. Cells were then allowed to grow for 24 hours in media containing 10% FBS, after which the cells were serum starved in 0.5% FBS media overnight. Cells were subsequently stimulated with fresh 0.5% FBS containing media and incubated under various experimental conditions for an additional 24 hours. Where indicated, cells were pre-incubated with specific compound inhibitors, for 20 minutes, prior to stimulation. Supernatants were collected and analyzed by ELISA (Cell Sciences).

### EXAMPLE 5

### siRNA Targeting hSK1 Decreases PAR-1-Induced Secretion of MCP-1

Our studies indicate a role for hSK1 in thrombin-mediated induction of MCP-1 expression. Therefore, we tested the ability of hSK1-specific siRNA to block secretion of MCP-1 protein from thrombin and PAR-1-AP stimulated HMVECs.

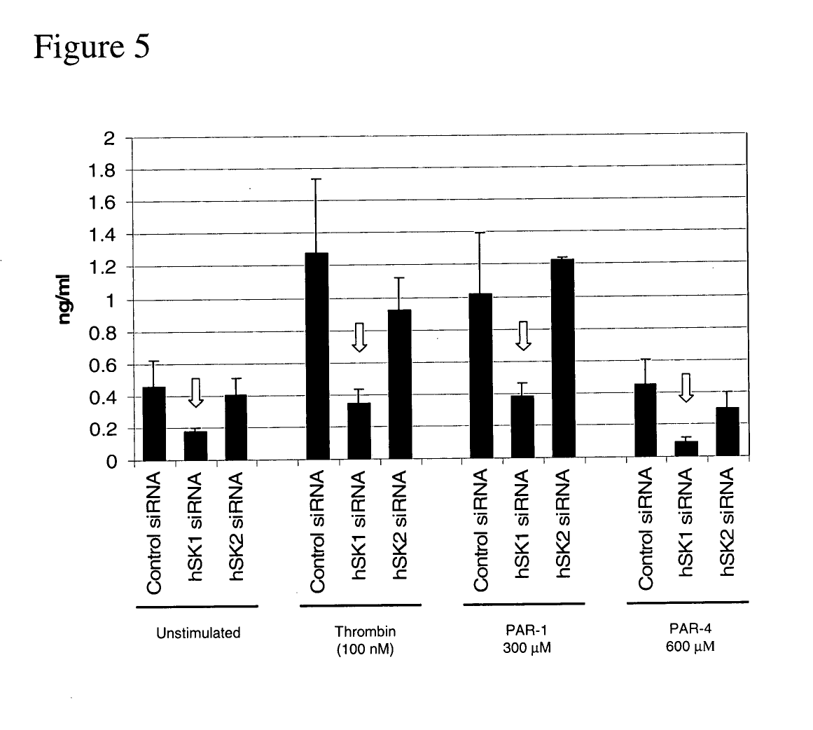

HMVECs were transfected with siRNAs (1.6 μg), hSK1-specific, hSK2-specific or control non-silencing siRNAs. After 48 hours, cells were stimulated with Thrombin (100 nM), PAR-1-AP (300 μM) or PAR-4-AP (600 μM). ELISA was performed to detect MCP-1 secretion into cell supernatant. As shown in FIG. 5, in the absence of hSK1 expression, as represented by hSK1-specific siRNA transfected cells (arrows), increased levels of MCP-1 secretion was inhibited. Furthermore, basal levels of MCP-1 protein secretion appeared to be effected as well, as the level of MCP-1 protein secretion in cells that were unstimulated or stimulated with PAR-4-AP was decreased from cells transfected with hSK1 specific siRNA.

### EXAMPLE 6

### Thrombin/PAR-1 or TNF-α-Mediated MCP-1 Protein Secretion Requires Both hSK and NF-κB Activity

To further characterize the signaling requirements of MCP-1 protein secretion from stimulated HMVECs, a panel of inhibitors to specific cell signaling components was used. The panel contained an inhibitor of hSK activity (DMS), an inhibitor of NF-κB activity (BAY11-7092), an inhibitor of PKC activity (GF109203x) and an ERK inhibitor (PD98059).

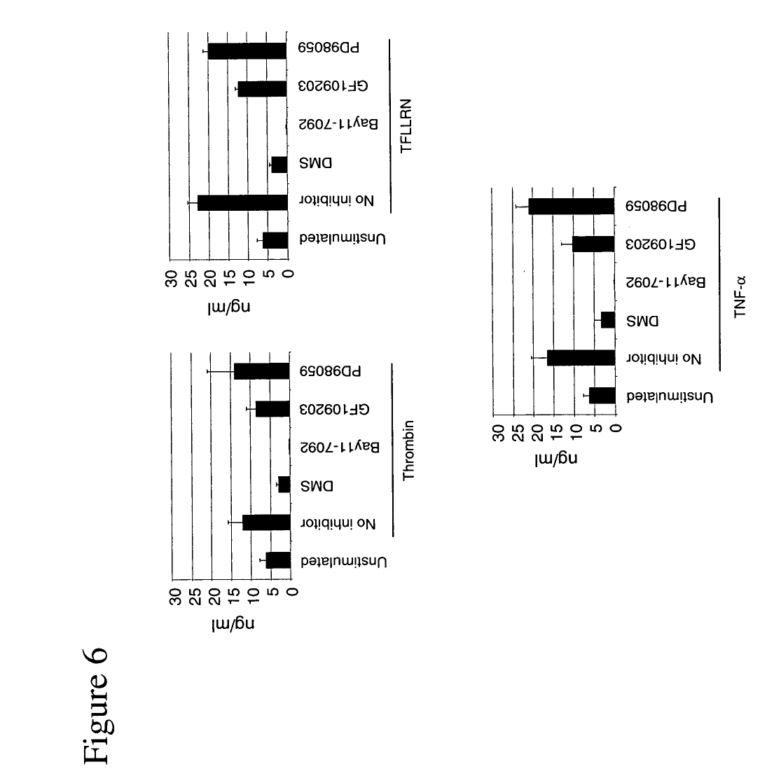

HMVECs were placed into culture overnight, and pretreated with inhibitors to hSK (DMS 10 μM), NF-κB (Bay11-7092 10 μM), PKC (GF109203x 1 μM), or Erk (PD98059 10 μM) prior to stimulation with Thrombin (100 nM), PAR-1-AP (TFLLRN 100 nM), or (Thrombin 0.1 ng/ml). ELISAs were performed 24 hours after stimulation to measure the amount of MCP-1 secretion. As shown in FIG. 6, we confirmed again that hSK activity is required for thrombin and PAR-1 mediated MCP-1 expression by ELISA. Furthermore we observed that NF-κB activity is a prerequisite for MCP-1 expression as well, as pretreatment with the NF-κB inhibitor, Bay11-7092, abrogated the induction of MCP-1 protein secretion by all agonists. While NF-κB activity was required for secretion of MCP-1 from HMVECs, we found that Erk activity was not required as pretreatment of the cells with PD98059 had no effect on secretion levels. PKC inhibition with GF109203x had an intermediate effect.

### EXAMPLE 6

### S1P Receptors are not Involved in MCP-1 Secretion

Recently, data has emerged that suggests the product of hSK activation, Sphingosine-1-phosphate, can be released from activated cells. As S1P is the ligand for a family of emerging GPCRs termed S1P receptors, we examined the potential of MCP-1 expression as a result of autocrine/paracrine S1P release on HMVECs.

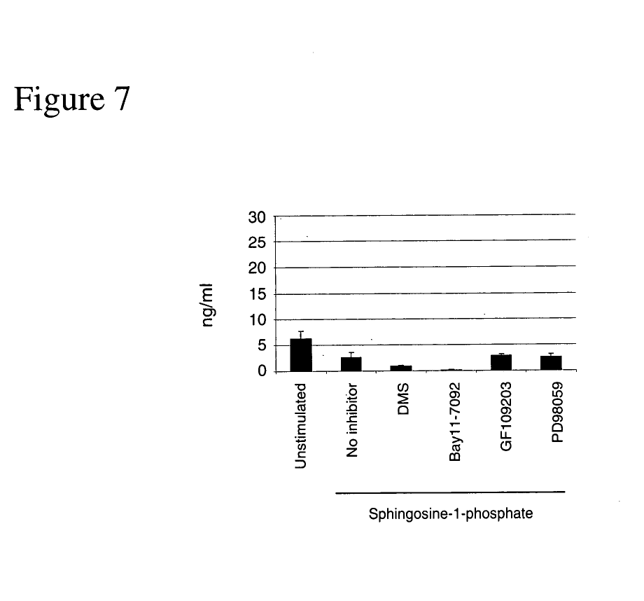

HMVECs were placed into culture overnight, and pretreated with inhibitors to hSK (DMS 10 μM), NF-κB (Bay11-7092 10 μM), PKC (GF109203×1 μM), or Erk (PD98059 10 μM) prior to stimulation with 5 μM S1P. ELISAs were performed to detect the levels of secreted MCP-1 protein. As illustrated in FIG. 7, 24 hours post S1P receptor activation by externally addition of S1P, the levels of MCP-1 being secreted is static. There was no elevation of MCP-1 protein by the external addition of S1P. Therefore, we rule out a role for S1P receptors in the hSK-dependent release of MCP-1 from HMVECs.

What is claimed is:

1. A method of determining a biological activity of a sphingosine kinase-1 in a cell, comprising the step of determining the expression level of a monocyte chemoattractant protein-1 gene from the cell.

2. A method of monitoring the effectiveness of a compound administered to a subject, wherein said compound is expected to increase or decrease the biological activity of a sphingosine kinase-1 in a cell of said subject, comprising the steps of:
a. measuring the expression level of a monocyte chemoattractant protein-1 gene from said subject; and
b. comparing the expression level determined in step a) with the expression level of a monocyte chemoattractant protein-1 gene in the subject prior to the administration of said compound.

3. The method of claim 2, further comprising the step of obtaining a biological sample from the subject, wherein the expression level of a monocyte chemoattractant protein-1 gene is determined from the biological sample.

4. The method of claim 3, wherein the biological sample from the subject comprises blood or plasma.

5. The method of claim 2, wherein the expression level of a monocyte chemoattractant protein-1 gene is determined by measuring the amount of mRNA of the monocyte chemoattractant protein-1 gene in the subject.

6. A method of monitoring the effectiveness of a compound administered to a subject, wherein said compound is expected to increase or decrease the biological activity of a sphingosine kinase-1 in a cell of said subject, comprising the steps of:
a. obtaining a test biological sample from the subject, wherein the test biological sample comprises blood or plasma from the subject;
b. measuring the amount of monocyte chemoattractant protein-1 in the test biological sample from the subject; and
c. comparing the amount of monocyte chemoattractant protein-1 determined in step a) with the amount of monocyte chemoattractant protein-1 in a control biological sample of the subject, wherein said control biological sample was obtained prior to the administration of said compound.

7. A kit for monitoring the effectiveness of a compound administered to a subject, wherein said compound is expected to increase or decrease the biological activity of a sphingosine kinase-1 in a cell of said subject, comprising
a. a nucleic acid probe that hybridizes under stringent hybridization condition to a monocyte chemoattractant protein-1 gene; and
b. an instruction for correlating the measured expressed level of monocyte chemoattractant protein-1 gene from said subject with the effectiveness of the compound.

8. A kit for monitoring the effectiveness of a compound administered to a subject, wherein said compound is expected to increase or decrease the biological activity of a sphingosine kinase-1 in a cell of said subject, comprising
a. an antibody that binds specifically a monocyte chemoattractant protein-1; and
b. an instruction for correlating the measured amount of monocyte chemoattractant protein-1 from said subject with the effectiveness of the compound.

9. A method of identifying a compound that increases or decreases the biological activity of a sphingosine kinase-1, comprising the steps of:
a. contacting a sphingosine kinase-1-responsive system with a solution comprising a buffer and a test compound, wherein the sphingosine kinase-1-responsive system comprises a sphingosine kinase-1 or a functional derivative thereof, and a gene whose expression is controlled by a regulatory sequence of a monocyte chemoattractant protein-1 gene;
b. measuring from the sphingosine kinase-1-responsive system the expression level of the gene whose expression is controlled by a regulatory sequence of a monocyte chemoattractant protein-1 gene; and
c. identifying the compound by its ability to increase or decrease said expression level as compared to a control wherein the sphingosine kinase-1-responsive system is contacted with only the buffer.

10. The method of claim 9, wherein the sphingosine kinase-1-responsive system is an animal, a tissue, or a cell.

11. The method of claim 9, wherein the sphingosine kinase-1-responsive system comprises a sphingosine kinase-1 that is expressed endogenously from the system.

12. The method of claim 9, wherein the sphingosine kinase-1-responsive system comprises a sphingosine kinase-1 that is expressed recombinantly from an exogenously DNA molecule introduced into system.

13. The method of claim 9, wherein the sphingosine kinase-1-responsive system comprises an endogenous monocyte chemoattractant protein-1.

14. The method of claim 9, wherein the sphingosine kinase-1-responsive system comprises a reporter gene whose expression is controlled by a regulatory sequence of a monocyte chemoattractant protein-1 gene.

15. The method of claim 14, wherein the reporter gene is selected from the group consisting of genes of green fluorescent protein (GFP), β-galactosidase (lacZ), luciferase (luc), chloramphenicol acetyltransferase (cat), β-glucuronidase, neomycin phosphotransferase, and guanine xanthine phosphoribosyl-transferase.

16. The method of claim 9, further comprising a step of contacting the sphingosine kinase-1-responsive system with a sphingosine kinase-1-activating stimulus, before the step (b) of claim 9.

17. The method of claim 16, wherein the sphingosine kinase-1-activating stimulus is thrombin or tumor necrosis factor alpha.

18. A method of identifying a compound that increases or decreases the biological activity of a sphingosine kinase-1, comprising the steps of:
a. contacting an endothelial cell with a test compound;
b. contacting the endothelial cell with a thrombin or tumor necrosis factor alpha;
c. measuring expression level of a monocyte chemoattractant protein-1 gene from the endothelial cell; and
d. identifying the compound by its ability to decrease monocyte chemoattractant protein-1 gene expression as compared to a control, wherein the endothelial cell is not contacted with a test compound.
wherein step (a) can be performed prior to, after, or simultaneously with step (b).

19. The method of claim 9 further comprising the steps of:
d. contacting a sphingosine kinase-1 with a solution comprising the compound identified from step (c) of claim 9 and a buffer comprising sphingosine and adenosine triphosphate;
e. measuring the amount of sphingosine-1-phosphate produced from the sphingosine; and
f. confirming the compound by its ability to increase or decrease the production of sphingosine-1-phosphate from the sphingosine as compared to a control wherein the sphingosine kinase-1 is contacted with only the buffer.

20. The method of claim 19, wherein the sphingosine kinase-1 is substantially purified.

21. A method of increasing or decreasing expression of a monocyte chemoattractant protein-1 gene in a cell, comprising the step of increasing or decreasing the biological activity of a sphingosine kinase-1 in the cell such that expression of said monocyte chemoattractant protein-1 gene is increased or decreased, respectively.

22. The method of claim 21, comprising the step of administering to the cell a compound that increases or decreases the catalytic activity of a sphingosine kinase-1 to form sphingosine-1-phosphate form sphingosine.

23. The method of claim 21, comprising the step of administering to the cell a compound that increases or decreases the expression of a sphingosine kinase-1 in the cell.

24. The method of claim 23, comprising the step of
a. introducing into the cell siRNA that targets the mRNA of a sphingosine kinase-1 gene for degradation;
b. maintaining the cell produced in (a) under conditions under which siRNA interference of the mRNA of the sphingosine kinase-1 gene in the cell occurs.

25. The method of claim 21, wherein said cell is an endothelial cell.

26. A method of preventing atherosclerosis in a subject comprising the step of decreasing the biological activity of a sphingosine kinase-1 in the subject such that atherosclerosis is prevented.

27. The method of claim 26, comprising the step of administering to the subject a compound that decreases the catalytic activity of a sphingosine kinase-1 to form sphingosine-1-phosphate from sphingosine.

28. The method of claim 21, comprising the step of administering to the cell a compound that decreases the expression of a sphingosine kinase-1 in the cell.

29. The method of claim 28, comprising the step of
a. introducing into a cell of the subject siRNA that targets the mRNA of a sphingosine kinase-1 gene for degradation;
b. maintaining the cell produced in (a) under conditions under which siRNA interference of the mRNA of the sphingosine kinase-1 gene in the cell occurs.

30. A method of treating atherosclerosis in a subject comprising the step of decreasing the biological activity of a sphingosine kinase-1 in the subject such that atherosclerosis is treated.

31. The method of claim 30, comprising the step of administering to the subject a compound that decreases the catalytic activity of a sphingosine kinase-1 to form sphingosine-1-phosphate from sphingosine.

32. The method of claim 30, comprising the step of administering to the cell a compound that decreases the expression of a sphingosine kinase-1 in the cell.

33. The method of claim 32, comprising the step of
a. introducing into a cell of the subject siRNA that targets the mRNA of a sphingosine kinase-1 gene for degradation;
b. maintaining the cell produced in (a) under conditions under which siRNA interference of the mRNA of the sphingosine kinase-1 gene in the cell occurs.

34. A method of inhibiting a thrombin signal transduction comprising the step of decreasing the biological activity of a sphingosine kinase-1 in the cell such that the thrombin signal transduction is inhibited.

35. The method of claim 34, wherein said signal transduction pathway involves a protease-activated receptor-1.

36. The method of claim 34, comprising the step of administering to the subject a compound that decreases the catalytic activity of a sphingosine kinase-1 to form sphingosine-1-phosphate from sphingosine.

37. The method of claim 34, comprising the step of administering to the cell a compound that decreases the expression of a sphingosine kinase-1 in the cell.

38. The method of claim 37, comprising the step of
a. introducing into a cell of the subject siRNA that targets the mRNA of a sphingosine kinase-1 gene for degradation;
b. maintaining the cell produced in (a) under conditions under which siRNA interference of the mRNA of the sphingosine kinase-1 gene in the cell occurs.

39. A method of preventing thrombosis in a subject comprising the step of decreasing the biological activity of a sphingosine kinase-1 in the subject such that thrombosis is prevented.

40. A method of treating thrombosis in a subject comprising the step of decreasing the biological activity of a sphingosine kinase-1 in the subject such that thrombosis is treated.
